(19) United States
(12) Patent Application Publication
(10) Pub. No.: US 2025/0250281 A1
(43) Pub. Date: Aug. 7, 2025

(54) PROCESS FOR PREPARATION OF RELUGOLIX

(71) Applicant: Glenmark Life Sciences Limited, Solapur (IN)

(72) Inventors: Venkata Raghavendra Charyulu PALLE, Pune (IN); Suresh KADAM, Thane (IN); Sukumar SINHA, Navi Mumbai (IN); Sachin GAVHANE, Thane (IN); Shailendra Nilkanth BHADANE, Navi Mumbai (IN); Uddhav Popat CHAUDHAR, Jalgaon (IN); Ajit Shankar MINDHE, Thane (IN); Namrata Govind RANE, Thane (IN); Nikhil Uttam SHELKE, Dombivli (IN)

(21) Appl. No.: 18/855,137

(22) PCT Filed: Apr. 5, 2023
(86) PCT No.: PCT/IB2023/053463

§ 371 Date: Oct. 8, 2024

(30) Foreign Application Priority Data
Apr. 8, 2022 (IN) 202221021078

(51) Int. Cl. C07D 495/04; C07D 333/38; C07D 409/12

(52) U.S. Cl. CPC C07D 495/04; C07D 333/38; C07D 409/12

(57) ABSTRACT

The present invention is related to a process for the preparation of relugolix, a compound of formula I, also known as N-[4-[1-[(2,6-difluoro phenyl) methyl]-5-[(dimethyl amino]-methyl]-3-(6-methoxy-3-pyridazinyl]-2,4-dioxo-1,2,3,4-tetrahydro-thieno[2,3-d]pyrimidin-6-yl]phenyl]-N′-methoxyurea, or a pharmaceutically acceptable salt thereof. The present invention is also related to a process for the preparation of crystalline relugolix.

### PRIORITY

This application claims the benefit of Indian Provisional Application No. 202221021078 filed on Apr. 8, 2022, entitled “Process for preparation of relugolix”, the contents of which are incorporated herein by reference.

### BACKGROUND OF THE INVENTION

### Technical Field

The present invention relates to a process for the preparation of relugolix. The present invention also relates to a process for the preparation of crystalline relugolix.

### Description of the Related Art

Relugolix, also known as N-[4-[1-[(2,6-difluorophenyl)methyl]-5-[(dimethylamino]-methyl]-3-(6-methoxy-3-pyridazinyl]-2,4-dioxo-1,2,3,4-tetrahydro-thieno[2,3-d]pyrimidin-6-yl]phenyl]-N′-methoxyurea, is represented by the structure of formula I.

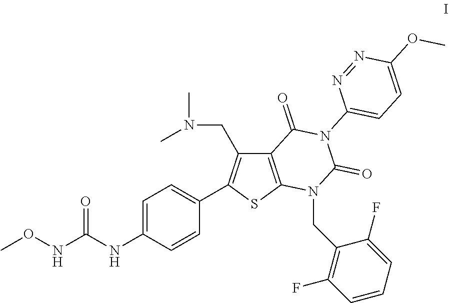

Relugolix is a gonadotropin-releasing hormone (GnRH) receptor antagonist indicated for the treatment of adult patients with advanced prostate cancer.

Relugolix is disclosed in published PCT Application No. WO 2004/067535A1 (the WO '535 publication).

The synthesis of Relugolix is described in the WO '535 publication and J. Med. Chem., 2011, 54 (14), pp. 4998-5012. Alternate process for the preparation of Relugolix is also described in Patent No. U.S. Pat. No. 9,758,528B2.

A combination of relugolix, a GnRH receptor antagonist, estradiol, an estrogen, and norethindrone acetate, a progestin, is indicated for the management of heavy menstrual bleeding associated with uterine leiomyomas (fibroids) in premenopausal women.

### SUMMARY OF THE INVENTION

The present invention provides a process for the preparation of relugolix, a compound of formula I (the “compound I”) or a pharmaceutically acceptable salt thereof,

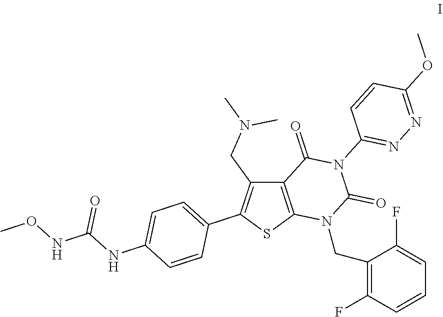 [SMILES: CONC(=O)Nc1ccc(-c2sc3c(c2CN(C)C)c(=O)n(-c2ccc(OC)nn2)c(=O)n3Cc2c(F)cccc2F)cc1]

the process comprising the steps of:
(a) reduction of a compound of formula XIX (the “compound XIX”) to obtain a compound of formula XVIII (the “compound XVIII”);

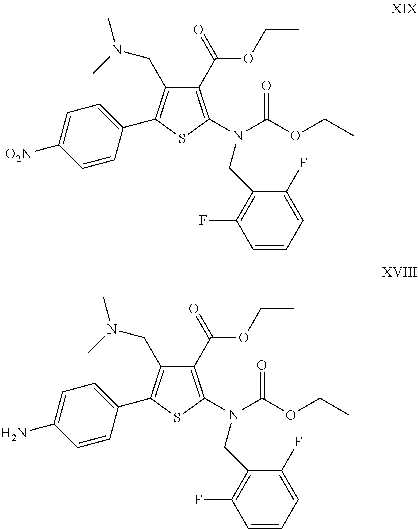 [SMILES: CCOC(=O)c1c(N(Cc2c(F)cccc2F)C(=O)OCC)sc(-c2ccc([N+](=O)[O-])cc2)c1CN(C)C ; CCOC(=O)c1c(N(Cc2c(F)cccc2F)C(=O)OCC)sc(-c2ccc(N)cc2)c1CN(C)C]

(b) reacting the compound XVIII with 1,1′-carbonyldiimidazole or a salt thereof, and methoxyamine or a salt thereof to obtain a compound of formula XVII (the “compound XVII”);

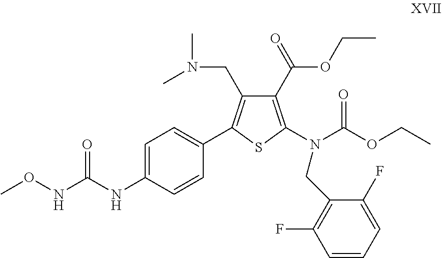 [SMILES: CCOC(=O)c1c(N(Cc2c(F)cccc2F)C(=O)OCC)sc(-c2ccc(NC(=O)NOC)cc2)c1CN(C)C]

(c) hydrolysing the compound XVII to obtain a compound of formula XVI (the “compound XVI”);

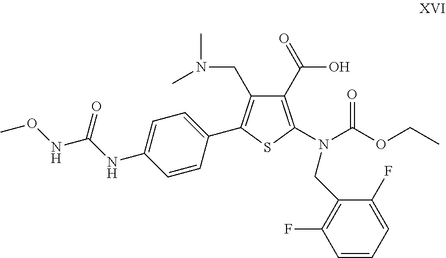 [SMILES: CCOC(=O)N(Cc1c(F)cccc1F)c1sc(-c2ccc(NC(=O)NOC)cc2)c(CN(C)C)c1C(=O)O]

(d) reacting the compound XVI with 3-amino-6-methoxypyridazine, a compound of formula VI (the “compound VI”) or a salt thereof, in the presence of a coupling agent to obtain a compound of formula XV (the “compound XV”);

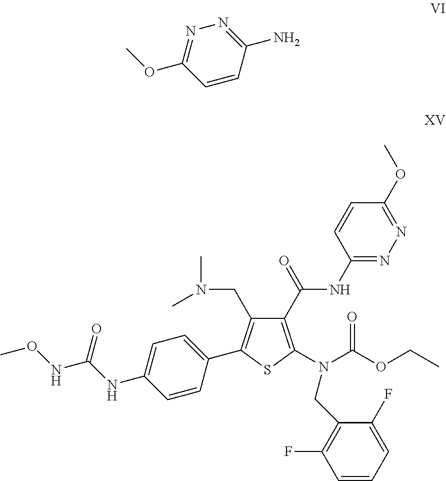

(e) cyclizing the compound XV in the presence of a base to obtain relugolix, the compound I, and optionally converting it to a pharmaceutically acceptable salt thereof; wherein the compound XVIII obtained in step (a), without isolation, is carried forward for reaction in the step (b).

The present invention also provides a process for the preparation of crystalline relugolix, the process comprising:
(1) providing a solution of relugolix, the compound I, in dimethylacetamide;
(2) combining the solution of the step (1) with an alcohol solvent to form a mixture;
(3) optionally, filtering the mixture of the step (2) to obtain a filtrate;
(4) obtaining crystalline relugolix from the mixture of the step (2) or the filtrate of the step (3); and
(5) isolating the crystalline relugolix, wherein the crystalline relugolix is characterized by an X-ray powder diffraction (XRPD) spectrum having peak reflections at about 7.3, 8.9, 9.9, 12.0, 16.6, 17.3, 22.2, 22.7, and 27.4±0.2 degrees 2 theta.

### BRIEF DESCRIPTION OF THE DRAWINGS

FIG. 1 is a characteristic X-ray Powder Diffraction Pattern (XRPD) of crystalline relugolix as obtained in Example 9.

FIG. 2 is a Differential Scanning Calorimetry (DSC) thermogram of crystalline relugolix as obtained in Example 9.

FIG. 3 is a Thermogravimetric Analysis (TGA) thermogram of crystalline relugolix as obtained in Example 9.

FIG. 4 is a characteristic X-ray Powder Diffraction Pattern (XRPD) of crystalline relugolix as obtained in Example 14.

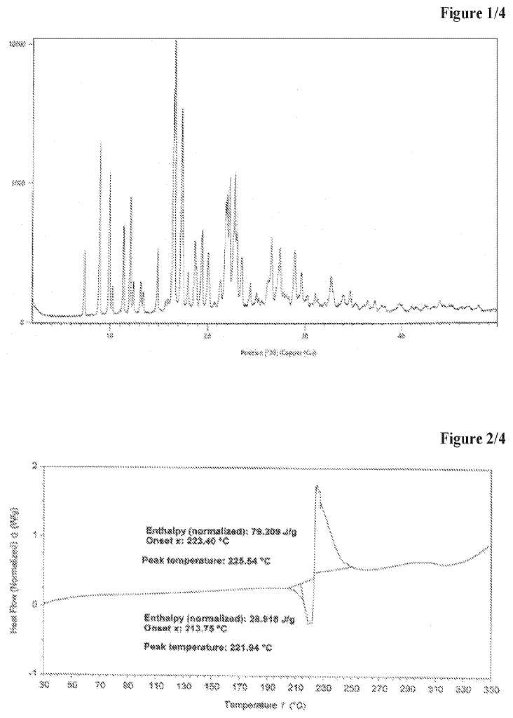

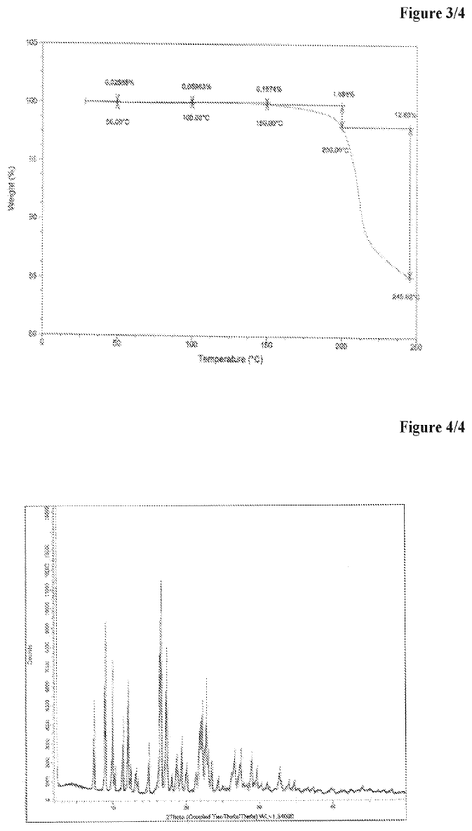

### DETAILED DESCRIPTION OF THE INVENTION

In one aspect, the present invention provides a process for the preparation of relugolix, a compound of formula I (the “compound I”) or a pharmaceutically acceptable salt thereof,

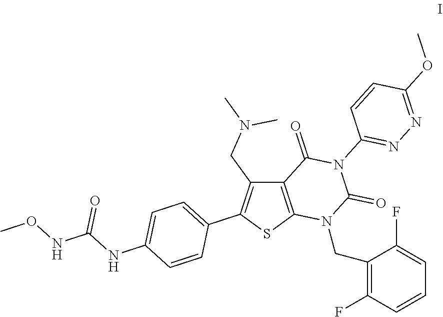 [SMILES: CONC(=O)Nc1ccc(-c2sc3c(c2CN(C)C)c(=O)n(-c2ccc(OC)nn2)c(=O)n3Cc2c(F)cccc2F)cc1]

the process comprising the steps of.
(a) reduction of a compound of formula XIX (the “compound XIX”) to obtain a compound of formula XVIII (the “compound XVIII”);

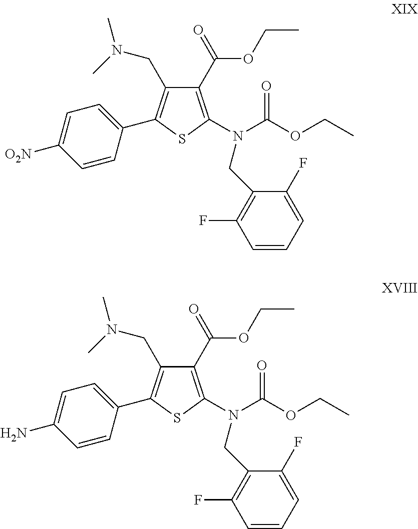 [SMILES: CCOC(=O)c1c(N(Cc2c(F)cccc2F)C(=O)OCC)sc(-c2ccc([N+](=O)[O-])cc2)c1CN(C)C ; CCOC(=O)c1c(N(Cc2c(F)cccc2F)C(=O)OCC)sc(-c2ccc(N)cc2)c1CN(C)C]

(b) reacting the compound XVIII with 1,1′-carbonyldiimidazole or a salt thereof, and methoxyamine or a salt thereof to obtain a compound of formula XVII (the “compound XVII”);

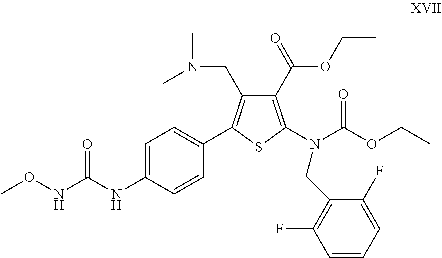 [SMILES: CCOC(=O)c1c(N(Cc2c(F)cccc2F)C(=O)OCC)sc(-c2ccc(NC(=O)NOC)cc2)c1CN(C)C]

(c) hydrolysing the compound XVII to obtain a compound of formula XVI (the “compound XVI”);

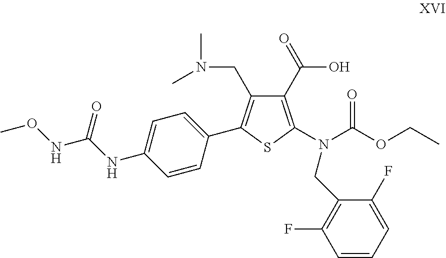 [SMILES: CCOC(=O)N(Cc1c(F)cccc1F)c1sc(-c2ccc(NC(=O)NOC)cc2)c(CN(C)C)c1C(=O)O]

(d) reacting the compound XVI with 3-amino-6-methoxypyridazine, a compound of formula VI (the “compound VI”) or a salt thereof, in the presence of a coupling agent to obtain a compound of formula XV (the “compound XV”);

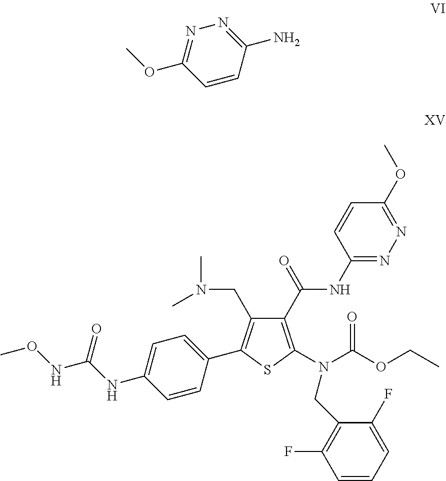

(e) cyclizing the compound XV in the presence of a base to obtain relugolix, the compound I, and optionally converting it to a pharmaceutically acceptable salt thereof; wherein the compound XVIII obtained in step (a), without isolation, is carried forward for reaction in the step (b).

In the context of the present invention, the term “room temperature” means a temperature of about 25° C. to about 30° C.

In one embodiment, as used herein, the term “about” refers to any value which lies within the range defined by a number up to 10% of the value.

In one embodiment, the reduction reaction of the step (a) is carried out by hydrogenation of the compound XIX in the presence of a catalyst.

In one embodiment, in the step (a), the catalyst is selected from the group consisting of palladium, platinum, Raney nickel, and a mixture thereof.

In one embodiment, the reduction reaction of step (a) is carried out by hydrogenation of the compound XIX in the presence of palladium as the catalyst.

In one embodiment, the palladium catalyst is supported on carbon support.

In one embodiment, the catalyst used is 5% palladium on carbon.

In one embodiment, the reduction reaction of the step (a) is carried out under the presence of hydrogen gas pressure.

In one embodiment, the hydrogen gas pressure used in the range from about is 1-5 kg/cm².

In one embodiment, the reduction reaction of the step (a) is carried out in the absence of an organic solvent.

In one embodiment, the reduction reaction of the step (a) is carried out in an aqueous medium.

In one embodiment, the aqueous medium comprises water.

In one embodiment, the reduction reaction of the step (a) further comprises addition of an organic acid.

In one embodiment, the organic acid is selected from the group consisting of formic acid, acetic acid, propionic acid, butanoic acid, trifluoroacetic acid, phthalic acid, fumaric acid, oxalic acid, tartaric acid, maleic acid, citric acid, succinic acid, malic acid, methanesulfonic acid, benzenesulfonic acid or p-toluenesulfonic acid and a mixture thereof.

In one embodiment, the organic acid used is acetic acid.

In one embodiment, in the step (a), the reaction may be carried out at a temperature of about 50° C. to about 100° C. The reaction time may range from about 30 minutes to about 10 hours, or longer.

In one embodiment, in the step (a), a biphasic reaction mixture is generated by adding a water immiscible organic solvent to the reaction mixture obtained after reduction of the compound XIX.

In one embodiment, in the step (a), the water immiscible organic solvent used for generating the biphasic reaction mixture is selected from the group consisting of an ester solvent, a haloalkane solvent, a hydrocarbon and a mixture thereof.

In one embodiment, the ester solvent is selected from methyl acetate, ethyl acetate, n-propyl acetate, n-butyl acetate or tert-butyl acetate.

In one embodiment, the ester solvent is ethyl acetate.

In one embodiment, the haloalkane solvent is selected from dichloromethane, chloroform, or dichloroethane.

In one embodiment, the haloalkane solvent is dichloromethane.

In one embodiment, the hydrocarbon is selected from heptane, hexane, pentane, cyclohexane, toluene, xylene.

In further embodiment, in the step (a), the biphasic reaction mixture obtained is treated with a base.

In one embodiment, the base is selected from the group consisting of lithium carbonate, sodium carbonate, potassium carbonate, ammonium carbonate, sodium bicarbonate, potassium bicarbonate, lithium bicarbonate, ammonium bicarbonate, sodium hydroxide, potassium hydroxide, lithium hydroxide, ammonium hydroxide and a mixture thereof.

In one embodiment, the base used is sodium bicarbonate.

In one embodiment, in the step (a), after treatment of the biphasic reaction mixture with the base, the biphasic reaction mixture is subjected to layer separation to obtain an organic layer and an aqueous layer, wherein the organic layer contains the compound XVIII.

In one embodiment, in the step (a), the organic layer containing the compound XVIII is not isolated and carried forward as such for further reaction in the step (b).

In the context of the present invention, the term “without isolation” as used herein means that the compound referred to is not separated as a solid, and that the compound remains in the solution.

In one embodiment, the compound XVIII obtained in step (a) is in-situ, and is carried forward to step (b).

In the context of the present invention, the term in-situ means the intermediate formed in the step referred to is not isolated.

In one embodiment, in the step (b) of the process for preparation of relugolix, the compound XVIII obtained in the step (a) is reacted in-situ with 1,1′-carbonyldiimidazole or a salt thereof, and methoxyamine or a salt thereof to obtain a compound XVII.

In one embodiment, in the step (b), the organic layer containing the compound XVIII obtained from step (a) is subjected to reaction with 1,1′-carbonyldiimidazole or a salt thereof, and methoxyamine or a salt thereof to obtain a compound XVII.

In one embodiment, the salt of 1,1′-carbonyldiimidazole includes but is not limited to 1,1′-carbonyldiimidazole hydrochloride.

In one embodiment, the salt of methoxyamine includes but is not limited to methoxyamine hydrochloride.

In one embodiment, the step (b) is carried out in the presence of a base.

In one embodiment, the base is selected from an organic base or an inorganic base.

In one embodiment, the organic base is selected from the group consisting of diisopropylethylamine, trimethylamine, triethylamine, tributylamine, triphenylamine, pyridine, lutidine, collidine, imidazole, DMAP (4-(dimethylamino)pyridine), DABCO (1,4-diazabicyclo[2.2.2]octane), DBU (1,8-diazabicyclo[5.4.0]undec-7-ene), DBN (1,5-diazabicyclo[4.3.0]non-5-ene), and N,N,N′,N′-tetramethyl-1,8-naphthalenediamine.

In one embodiment, the inorganic base is selected from the group consisting of lithium carbonate, sodium carbonate, potassium carbonate, sodium bicarbonate, potassium bicarbonate, and a mixture thereof.

In one embodiment, in the step (b), the base used is triethylamine.

In one embodiment, in the step (b), the reaction may be carried out at a temperature of about 20° C. to about 80° C.

In one embodiment, in the step (b), the reaction may be stirred for a suitable time.

The stirring time may range from about 30 minutes to about 10 hours, or longer.

In one embodiment, in the step (b), a biphasic reaction mixture is generated by adding water to the reaction mixture after completion of the reaction.

In one embodiment, in the step (b), the biphasic reaction mixture is subjected to layer separation to obtain an organic layer and an aqueous layer, wherein the organic layer contains the compound XVII.

In one embodiment, the compound XVII obtained in the step (b) is not isolated and carried forward as such for further reaction in step (c).

In one embodiment, in the step (b), the organic layer is concentrated under reduced pressure to obtain the compound XVII.

In one embodiment, in the step (c) of the process for the preparation of relugolix (the compound I), the compound XVII is hydrolyzed to obtain the compound XVI.

In one embodiment, the reaction of step (c) involving hydrolysis is carried out in the presence of a base.

In one embodiment, the base is selected from the group consisting of lithium hydroxide, sodium hydroxide, potassium hydroxide, ammonium hydroxide, sodium methoxide, sodium ethoxide and a mixture thereof.

In one embodiment, the base used in step (c) is sodium hydroxide.

In one embodiment, the reaction of step (c) involving hydrolysis is carried out in the presence of an acid.

In one embodiment, the acid is selected from the group consisting of hydrochloric acid, sulphuric acid, nitric acid, formic acid, acetic acid, trifluoroacetic acid, and a mixture thereof.

In one embodiment, in the step (c), the hydrolysis reaction is carried out in the presence of a solvent.

In one embodiment, the solvent is selected from the group consisting of an alcohol such as methanol, ethanol, 1-propanol, 2-propanol, 1-butanol, t-butanol, pentanol and the like; an amide such as dimethylacetamide, dimethylformamide, and the like; ether such as tetrahydrofuran, methyl tert-butyl ether, 1,4-dioxane and the like; a nitrile such as acetonitrile, propionitrile, butyronitrile and the like, a ketone such as acetone, ethyl methyl ketone, methyl isobutyl ketone and the like; dimethylsulfoxide; N-methylpyrrolidone; water; and a mixture thereof.

In one embodiment, the solvent used in the step (c) is an alcohol.

In one embodiment, the solvent used in the step (c) is methanol.

In one embodiment, in the step (c), the reaction may be carried out at a temperature of about 20° C. to about 100° C.

In one embodiment, in the step (c), the reaction may be stirred for a suitable time. The stirring time may range from about 30 minutes to about 10 hours, or longer.

In one embodiment, the compound XVI obtained in the step (c) is subjected to base-acid treatment.

In one embodiment, the base is selected from the group consisting of sodium hydroxide, potassium hydroxide, lithium hydroxide, and a mixture thereof. In an embodiment, the base used is sodium hydroxide (NaOH).

In one embodiment, the acid is selected from the group consisting of hydrochloric acid, hydrobromic acid, acetic acid, and a mixture thereof. In an embodiment, the acid used is hydrochloric acid (HCl).

In one embodiment, the compound XVI obtained in the step (c) is subjected to NaOH—HCl treatment.

In one embodiment, in the step (c), the compound XVI is obtained in a purity of ≥98.5% and wherein the level of impurity A, impurity B, impurity C, or impurity D represented by the following structural formulae is less than 0.5% w/w relative to the amount of the compound of formula XVI, as determined by High Performance Liquid Chromatography (HPLC),

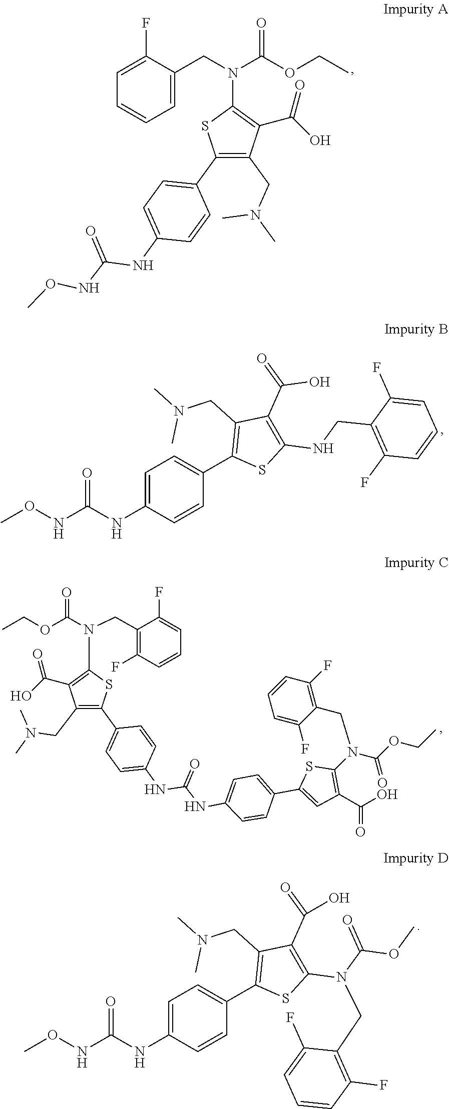

In one embodiment, the compound XVI is obtained in a purity of ≥98.5%, and wherein the level of any of the impurity as described above is less than 0.15%.

In one embodiment, the compound XVI is obtained in a purity of ≥98.5%, and wherein the level of any of the impurity as described above is less than 0.05%.

In one embodiment, in the step (d) of the process for preparation of relugolix (the compound I), the compound XVI is reacted with 3-amino-6-methoxypyridazine or a salt thereof, in the presence of a coupling agent to obtain the compound XV.

In one embodiment, the salt of 3-amino-6-methoxypyridazine includes but is not limited to 3-amino-6-methoxypyridazine hydrochloride.

In one embodiment, in the step (d), the coupling agent is selected from the group consisting of a carbodiimide reagent, an anhydride reagent, a benzotriazole reagent, a phosphorus reagent, a borate reagent, a quinoline reagent, an oxime reagent, and a mixture thereof.

In one embodiment, the coupling agent is carbodiimide reagent, which includes, but is not limited to EDCI (N-(3-dimethylaminopropyl)-N′-ethylcarbodiimide hydrochloride), DCC (dicyclohexylcarbodiimide), DIC (diisopropylcarbodiimide) and the like.

In one embodiment, the coupling agent is anhydride reagent, which includes, but is not limited to T₃P (propylphosphonic anhydride) and the like.

In one embodiment, the coupling agent is benzotriazole reagent, which includes but is not limited to HBTU (N,N,N′,N′-tetramethyl-O-(1H-benzotriazol-1-yl)uronium hexafluorophosphate), HOBt (hydroxybenzotriazole hydrate), TBTU (O-(benzotriazol-1-yl)-N,N,N′,N′-tetramethyluronium tetrafluoroborate), TATU (O-(7-azabenzotriazole-1-yl)-1,1,3,3-tetramethyluronium tetrafluoroborate), PyBOP ((benzotriazol-1-yloxy) tripyrrolidinophosphonium hexafluorophosphate), TDBTU (O-(3,4-dihydro-4-oxo-1,2,3-benzotriazin-3-yl)-N,N,N′,N′-tetramethyluronium tetrafluoroborate), HDMC (N-[(5-Chloro-3-oxido-1H-benzotriazol-1-yl)-4-morpholinylmethylene]-N-methyl-methanaminium hexafluorophosphate), HCTU (2-(6-chloro-1H-benzotriazole-1-yl)-1,1,3,3-tetramethylaminium hexafluorophosphate), DEPBT (3-(diethoxyphosphoryloxy)-1,2,3-benzotriazin-4(3H)-one), PyAOP ((7-azabenzotriazol-1-yloxy)tripyrrolidinophosphonium hexafluorophosphate), PyBOP (benzotriazol-1-yl-oxytripyrrolidinophosphonium hexafluorophosphate), BOP (benzotriazol-1-yloxytris(dimethylamino)phosphonium hexafluorophosphate), HOOBt (hydroxy-3,4-dihydro-4-oxo-1,2,3-benzotriazine), HOSu (N-hydroxysuccinimide), HOAt (1-hydroxy-7-azabenzotriazole) and the like.

In one embodiment, the coupling agent is phosphorus reagent, which includes but is not limited to COMU ((1-cyano-2-ethoxy-2-oxoethylidenaminooxy)dimethylamino-morpholino-carbenium hexafluorophosphate), HOTT (S-(1-oxido-2-pyridyl)-N,N,N′,N′-tetramethylthiuronium hexafluorophosphate), PyCIU (chlorodipyrrolidinocarbenium hexafluorophosphate), TFFH (tetramethylfluoroformamidinium hexafluorophosphate), FDPP (pentafluorophenyl diphenylphosphinate) and the like.

In one embodiment, the coupling agent is borate reagent, which includes but is not limited to DMTMM (4-(4,6-dimethoxy-1,3,5-triazin-2-yl)-4-methylmorpholinium tetrafluoroborate), TSTU (N,N,N,N-tetramethyl-O—(N-succinimidyl)uronium tetrafluoroborate), TPTU (O-(2-oxo-1 (2H)pyridyl)-N,N,N′,N′-tetramethyluronium tetrafluoroborate), TOTU (O-[(ethoxycarbonyl)cyanomethylenamino]-N,N,N′,N′-tetramethyluronium tetrafluoroborate) and the like.

In one embodiment, the coupling agent is quinoline reagent, which includes but is not limited to IIDQ (isobutyl 1,2-dihydro-2-isobutoxy-1-quinolinecarboxylate), EEDQ (N-Ethoxycarbonyl-2-ethoxy-1,2-dihydroquinoline) and the like.

In one embodiment, the coupling agent is oxime reagent, which includes but is not limited to Oxyma (ethyl (hydroxyimino)cyanoacetate), PyOxim ([ethyl cyano(hydroxyimino)acetato-O2]tri-1-pyrrolidinylphosphonium hexafluorophosphate), and the like.

In one embodiment, the coupling agent used in the step (d) is propylphosphonic anhydride (T₃P).

In one embodiment, in the step (d), the reaction of the compound XVI with the compound VI is carried out in the presence of a base.

In one embodiment, in the step (d), the base is selected from an organic base or an inorganic base.

In one embodiment, in the step (d), the organic base is selected from the group consisting of diisopropylethylamine, trimethylamine, triethylamine, tributylamine, triphenylamine, pyridine, lutidine, collidine, imidazole, DMAP (4-(dimethylamino)pyridine), DABCO (1,4-diazabicyclo[2.2.2]octane), DBU (1,8-diazabicyclo[5.4.0]undec-7-ene), DBN (1,5-diazabicyclo[4.3.0]non-5-ene), N,N,N′,N′-tetramethyl-1,8-naphthalenediamine and a mixture thereof.

In one embodiment, in the step (d), the inorganic base is selected from the group consisting of lithium carbonate, sodium carbonate, potassium carbonate, sodium bicarbonate, potassium bicarbonate, and a mixture thereof.

In one embodiment, in the step (d), the base used is diisopropylethylamine.

In one embodiment, in the step (d), the reaction of the compound XVI with the compound VI is carried out in the presence of a solvent.

In one embodiment, in the step (d), the solvent is selected from the group consisting of an ester such as methyl acetate, ethyl acetate, isopropyl acetate and the like; an amide such as dimethylacetamide, dimethylformamide, and the like; an ether such as tetrahydrofuran, 1,4-dioxane and the like; a nitrile solvent such as acetonitrile, propionitrile, butyronitrile and the like; a haloalkane such as dichloromethane, dichloroethane, chloroform and the like; a ketone such as acetone, methyl isobutyl ketone and the like; dimethylsulfoxide; N-methylpyrrolidone; sulfolane; diglyme; and a mixture thereof.

In one embodiment, in the step (d), the solvent used is ester.

In one embodiment, in the step (d), the solvent used is ethyl acetate.

In one embodiment, the reaction of step (d) is carried out under inert atmosphere, such as under nitrogen or argon.

In one embodiment, the reaction of step (d) is carried out under nitrogen atmosphere.

In one embodiment, the reaction of step (d) may be carried out at a temperature of about 20° C. to about 70° C. The stirring time may range from about 30 minutes to about 6 hours, or longer.

In one embodiment, in the step (e) of the process for the preparation of relugolix (the compound I), the compound XV is cyclized in the presence of a base to obtain relugolix, the compound I, which may be optionally converted to a pharmaceutically acceptable salt thereof.

In one embodiment, in the step (e), the base is selected from the group consisting of sodium hydroxide, potassium hydroxide, lithium hydroxide, sodium methoxide, sodium ethoxide, potassium methoxide, ammonium hydroxide, sodium bicarbonate, potassium bicarbonate, sodium carbonate, potassium carbonate, calcium carbonate and a mixture thereof.

In one embodiment, in the step (e), the base used is sodium methoxide.

In one embodiment, in the step (e), the cyclization reaction is carried out in the presence of a solvent.

In one embodiment, the solvent is selected from the group consisting of an alcohol such as methanol, ethanol, 1-propanol, 2-propanol, n-butanol, t-butanol and the like; an ether such as tetrahydrofuran, dioxane and the like, a nitrile such as acetonitrile, propionitrile, butyronitrile and the like, an amide such as N,N-dimethylsulfoxide, N,N-dimethylacetamide, N,N-dimethyl formamide and the like, a haloalkane such as dichloromethane, dichloroethane, chloroform and the like, water or a mixture thereof.

In one embodiment, in the step (e), the solvent used is an alcohol.

In one embodiment, in the step (e), the solvent used is methanol.

In one embodiment, the reaction of the step (e) may be carried out at a temperature of about 40° C. to about 100° C.

In one embodiment, in the step (e), the reaction may be stirred for a suitable time.

The stirring time may range from about 30 minutes to about 8 hours, or longer.

In one embodiment, relugolix, the compound I, obtained in step (e) is treated with water and stirred. The stirring may be carried out at a temperature of about 20° C. to about 40° C. The stirring time may range from about 30 minutes to about 4 hours, or longer.

In another aspect, the present invention provides a process for the preparation of relugolix, a compound of formula I,

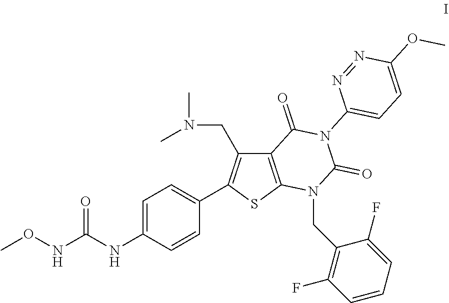

the process comprising:
(a-1) reacting a compound of formula III (the “compound III”) with a chlorinating agent to give a compound of formula II (the “compound II”); and

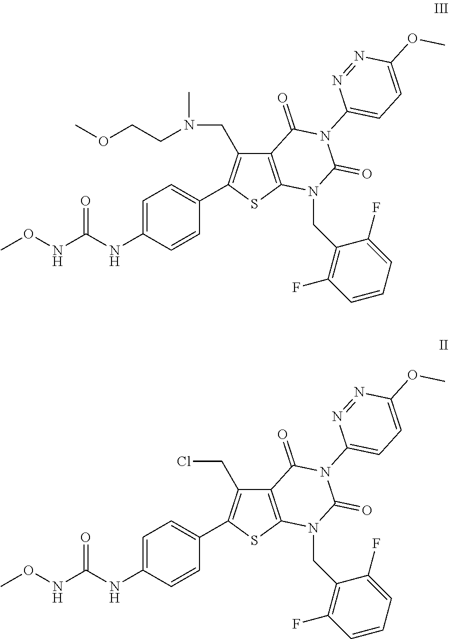

(b-1) reacting the compound II with dimethylamine to obtain relugolix, the compound I.

In an embodiment of the invention, the step (a-1) of the process for the preparation of relugolix, the compound I, the compound III is reacted with a chlorinating agent to give the compound of formula II.

In one embodiment, the chlorinating agent used in the step (a-1) includes, but is not limited to, 1-chloroethylchloroformate, 2-chloroethylchloroformate, N-chlorosuccinimide, thionyl chloride, phosphorus oxychloride, phosphorus trichloride, and the like.

In one embodiment, the chlorinating agent is 1-chloroethylchloroformate, a compound of formula IV,

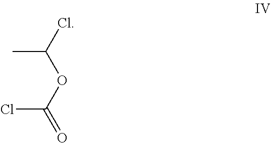

In one embodiment, the reaction in the step (a-1) is carried out such that the chlorinating agent may be added at a temperature of about −80° C. to about 0° C.

In one embodiment, in the step (a-1), the reaction is carried out in the presence of an organic solvent.

In one embodiment, the organic solvent includes, but is not limited to, haloalkanes such as dichloromethane, chloroform, ethylene dichloride and the like; ketones such as acetone, ethyl methyl ketone, methyl isobutyl ketone and the like; alcohols such as methanol, ethanol, n-propyl alcohol, isopropyl alcohol, n-butyl alcohol, isobutyl alcohol, tert-butyl alcohol, n-pentyl alcohol, n-octyl alcohol and the like; ethers such as dimethyl ether, diethyl ether, diisopropyl ether, tert-butyl methyl ether, dimethoxyethane, tetrahydrofuran, dioxane and the like; esters such as methyl acetate, ethyl acetate, n-propyl acetate, tert-butyl acetate and the like; hydrocarbons such as heptane, hexane, pentane, cyclohexane, toluene, xylene, and the like; amides such as dimethylformamide, dimethylacetamide and the like; sulfoxides such as dimethylsulfoxide; and mixtures thereof.

In one embodiment, in the step (a-1) of the process, on completion of addition of the chlorinating agent, the reaction mixture is stirred at a temperature of about 0° C. to about 40° C. The stirring time may range from about 30 minutes to about 10 hours, or longer.

In one embodiment, in the step (a-1), the compound II is isolated from the reaction mixture as a solid.

In one embodiment, in the step (b-1), the reaction is carried out in the presence of an organic solvent.

In one embodiment, the organic solvent includes, but is not limited to, haloalkanes such as dichloromethane, chloroform, ethylene dichloride and the like; ketones such as acetone, ethyl methyl ketone, methyl isobutyl ketone and the like; alcohols such as methanol, ethanol, n-propyl alcohol, isopropyl alcohol, n-butyl alcohol, isobutyl alcohol, tert-butyl alcohol, n-pentyl alcohol, n-octyl alcohol and the like; ethers such as dimethyl ether, diethyl ether, diisopropyl ether, tert-butyl methyl ether, dimethoxyethane, tetrahydrofuran, dioxane and the like; esters such as methyl acetate, ethyl acetate, n-propyl acetate, tert-butyl acetate and the like; hydrocarbons such as heptane, hexane, pentane, cyclohexane, toluene, xylene, and the like; amides such as dimethylformamide, dimethylacetamide and the like; sulfoxides such as dimethyl sulfoxide; and mixtures thereof.

In one embodiment, the present invention provides a process for the preparation of the compound III, the process comprising the steps of:
(i) reducing a compound of formula X (the “compound X”) to obtain a compound of formula IX (the “compound IX”);

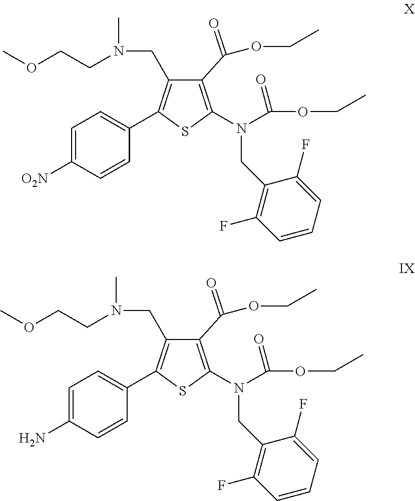

(ii) reacting the compound IX with 1,1′-carbonyldiimidazole or a salt thereof, and methoxyamine or a salt thereof to obtain a compound of formula VIII (the “compound VIII”);

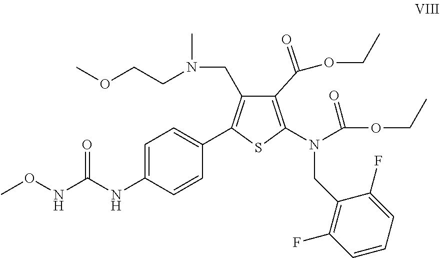 [SMILES: CCOC(=O)c1c(N(Cc2c(F)cccc2F)C(=O)OCC)sc(-c2ccc(NC(=O)NOC)cc2)c1CN(C)CCOC]

(iii) hydrolysing the compound VIII to obtain a compound of formula VII (the “compound VII”);

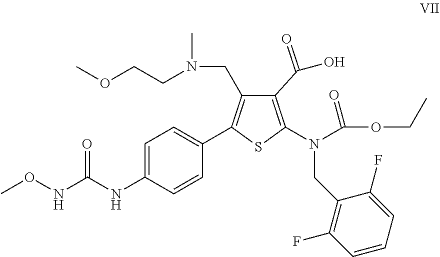 [SMILES: CCOC(=O)N(Cc1c(F)cccc1F)c1sc(-c2ccc(NC(=O)NOC)cc2)c(CN(C)CCOC)c1C(=O)O]

(iv) reacting the compound VII with 3-amino-6-methoxypyridazine, a compound of formula VI (the “compound VI”) or a salt thereof, in the presence of a coupling agent to obtain a compound of formula V (the “compound V”); and

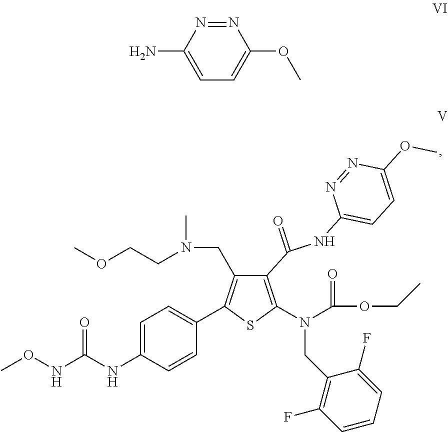

(v) cyclizing the compound V in the presence of a base to obtain the compound III.

In one embodiment, the reduction reaction in the step (i) of the above process, is carried out by hydrogenation of the compound X in the presence of a catalyst.

In one embodiment, the catalyst used in the step (i) is selected from the group consisting of palladium, platinum, Raney nickel, and mixtures thereof.

In one embodiment, the compound IX obtained in the step (i) is not isolated and carried forward as such for further reaction.

In one embodiment, the compound VIII obtained in the step (ii) is not isolated and carried forward as such for further reaction.

In one embodiment, in the step (iii), the hydrolysis of the compound VIII is carried out in the presence of a base to obtain the compound VII.

In one embodiment, the base includes, but is not limited to, sodium hydroxide, potassium hydroxide, lithium hydroxide, aqueous ammonia and the like.

In one embodiment, in the step (iv), the coupling agent selected is as discussed supra.

In one embodiment, the compound V obtained in the step (iv) is not isolated and carried forward as such for further reaction.

In one embodiment, the base used in the step (v) includes, but is not limited to, sodium hydroxide, potassium hydroxide, lithium hydroxide, sodium methoxide, potassium methoxide, aqueous ammonia and the like.

In one embodiment, the present invention provides a process for the preparation of the compound X, the process comprising the steps of.
(A) reacting a compound of formula XIV (the “compound XIV”) with ethyl haloformate in the presence of an organic solvent to obtain a compound of formula XIII (the “compound XIII”);

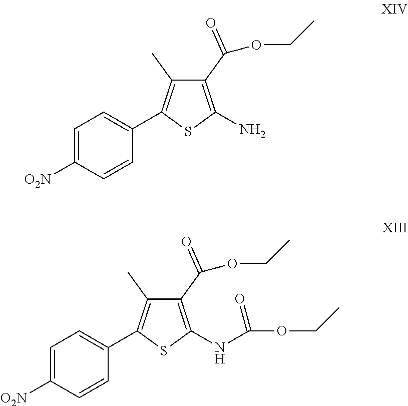 [SMILES: CCOC(=O)c1c(N)sc(-c2ccc([N+](=O)[O-])cc2)c1C ; CCOC(=O)Nc1sc(-c2ccc([N+](=O)[O-])cc2)c(C)c1C(=O)OCC]

(B) reacting the compound XIII with 2,6-difluorobenzyl halide to obtain a compound of formula XII (the “compound XII”);

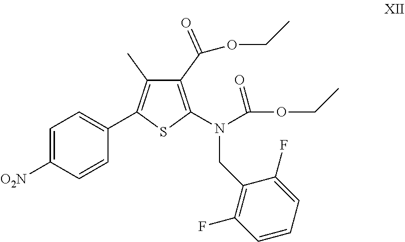 [SMILES: CCOC(=O)c1c(N(Cc2c(F)cccc2F)C(=O)OCC)sc(-c2ccc([N+](=O)[O-])cc2)c1C]

(C) brominating the compound XII in the presence of a brominating agent to obtain a compound of formula XI (the “compound XI”); and

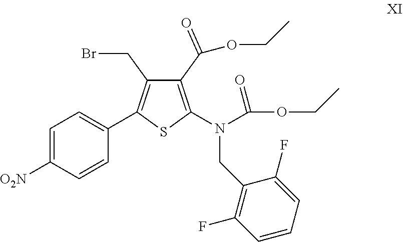 [SMILES: CCOC(=O)c1c(N(Cc2c(F)cccc2F)C(=O)OCC)sc(-c2ccc([N+](=O)[O-])cc2)c1CBr]

(D) reacting the compound XI with N-(2-methoxylethyl) methylamine to obtain the compound X.

In one embodiment, the ethyl haloformate used in step (A) is selected from ethyl chloroformate, or ethyl bromoformate.

In one embodiment, the 2,6-difluorobenzyl halide used in step (B) is selected from 2,6-difluorobenzyl chloride or 2,6-difluorobenzyl bromide.

In one embodiment, the brominating agent used in step (C) of the process includes, but is not limited to, bromine, N-bromosuccinimide, or a mixture thereof.

In one embodiment, the brominating agent is N-bromosuccinimide.

In one embodiment, the present invention provides a process for the preparation of the crystalline form of relugolix, the compound I, characterized by an X-ray powder diffraction (XRPD) spectrum having peak reflections at about 7.3, 8.9, 9.9, 12.0, 16.6, 17.3, 22.2, 22.7, and 27.4±0.2 degrees 2 theta, the process comprising crystallization of relugolix, the compound I, from a mixture of solvents comprising of ethyl acetate and tetrahydrofuran.

In one embodiment, the present invention provides crystalline relugolix, the compound I, characterized by XRPD spectrum having peak reflections as discussed supra, wherein the crystalline relugolix is prepared by a process comprising the steps of.
(a-i) dissolving relugolix, the compound I, in an organic solvent to form a solution;
(b-i) optionally, adding a second solvent to the solution of the step (a-i) to form a mixture;
(c-i) precipitating out relugolix, the compound I, from the solution of the step (a-i) or the mixture of the step (b-i); and
(d-i) isolating relugolix obtained in the step (c-i).

In one embodiment, the organic solvent used in the step (a-i) includes, but is not limited to, esters such as methyl acetate, ethyl acetate, n-propyl acetate, tert-butyl acetate and the like; alcohols such as methanol, ethanol, n-propyl alcohol, isopropyl alcohol, n-butyl alcohol, isobutyl alcohol, sec-butyl alcohol, tert-butyl alcohol, n-pentyl alcohol, n-octyl alcohol and the like, ketones such as acetone, ethyl methyl ketone, methyl isobutyl ketone and the like, ethers such as dimethyl ether, diethyl ether, diisopropyl ether, methyl tert-butyl ether, tetrahydrofuran, dioxane and the like; haloalkanes such as dichloromethane, chloroform, ethylene dichloride, and the like; sulfoxides such as dimethyl sulfoxide, amides such as dimethylformamide, dimethylacetamide and the like; N-methylpyrrolidone; water; or mixtures thereof.

In one embodiment, suitable temperature for dissolution of relugolix, the compound I, as per the step (a-i) may range from about 20° C. to about 120° C. Stirring may be continued for any desired time period to achieve a complete dissolution of relugolix. The stirring time may range from about 30 minutes to about 10 hours, or longer. The solution may be optionally treated with charcoal and filtered to get a particle-free solution.

In one embodiment, in the step (b-i), the second solvent includes, but is not limited to, hydrocarbons such as hexane, heptane and the like; water; or mixtures thereof.

In one embodiment, in the step (c-i), relugolix, the compound I, is precipitated out by stirring the solution of step (a-i) or the mixture of step (b-i). The stirring time may range from about 30 minutes to about 5 hours, or longer. The temperature may range from about 0° C. to about 85° C.

In the step (d-i) of the above process, relugolix, the compound I, is isolated from the solution by any method known in the art. The method, may involve any of the techniques, known in the art, including filtration by gravity or by suction, centrifugation, and the like.

In one embodiment, the present invention provides a process for the preparation of crystalline relugolix, the process comprising:
(1) providing a solution of relugolix in dimethylacetamide;
(2) combining the solution of the step (1) with an alcohol solvent to form a mixture;
(3) optionally, filtering the mixture of the step (2) to obtain a filtrate;
(4) obtaining crystalline relugolix from the mixture of step (2) or the filtrate of step (3); and
(5) isolating the crystalline relugolix, wherein the crystalline relugolix is characterized by an X-ray powder diffraction (XRPD) spectrum having peak reflections at about 7.3, 8.9, 9.9, 12.0, 16.6, 17.3, 22.2, 22.7, and 27.4±0.2 degrees 2 theta.

In one embodiment, the step (1) is carried out at a temperature of about 35° C. to about 60° C.

In one embodiment, the step (1) is carried out at a temperature of about 40° C. to about 45° C.

In one embodiment, in step (1), the solution may be stirred for a suitable time. Stirring may be continued for any desired time period to achieve a complete dissolution of relugolix.

In one embodiment, in the step (2), the alcohol solvent is selected from C₁-C₆ alkyl alcohol.

In one embodiment, the C₁-C₆ alkyl alcohol is selected from the group consisting of methanol, ethanol, 1-propanol, 2-propanol, n-butanol, iso-butanol, 2-butanol, tert-butanol, pentanol, and the like.

In the context of the present invention, the term “combining” means adding the solution of the step (1) to an alcohol solvent, or adding an alcohol solvent to the solution of the step (1).

In one embodiment, the step (2) comprises adding the solution of the step (1) to C₁-C₆ alkyl alcohol or adding C₁-C₆ alkyl alcohol to the solution of the step (1).

In one embodiment, the step (2) is carried out at a temperature of about 35° C. to about 60° C.

In one embodiment, the step (2) is carried out at a temperature of about 40° C. to about 45° C.

In one embodiment, in the step (3), filtration is carried out at a temperature same as that of step (2).

In one embodiment, in the step (3), filtration is carried out at a temperature of about 35° C. to about 60° C.

In one embodiment, in the step (3), filtration is carried out at a temperature of about 40° C. to about 45° C.

In one embodiment, in the step (3), filtration is carried out to get a particle-free filtrate.

In one embodiment, the step (4) of obtaining crystalline relugolix comprises cooling the mixture of the step (2) or the filtrate of the step (3).

In one embodiment, in the step (4), the cooling is carried out to a temperature of below about 30° C.

In the context of the present invention, the term “below about 30° C.” as used herein means the temperature ranging from about 30° C. to about 15° C., preferably about 30° C. to about 20° C., more preferably about 30° C. to about 25° C.

In one embodiment, in the step (4), the mixture of step (2) or the filtrate of the step (3) is cooled to a temperature of about 30° C. to about 20° C.

In one embodiment, in the step (4), the mixture of the step (2) or the filtrate of the step (3) is cooled to a temperature of about 30° C. to about 25° C.

In the step (5) of the above process, relugolix, the compound I, is isolated from the solution by any method known in the art. The method, may involve any of the techniques, known in the art, including filtration by gravity or by suction, centrifugation, and the like.

In one embodiment, the present invention provides a process for the preparation of crystalline relugolix, the process comprising the steps of
(1) providing a solution of relugolix in dimethylacetamide at a temperature of about 35° C. to about 60° C.;
(2) combining the solution of the step-(1) with an alcohol solvent at a temperature of about 35° C. to about 60° C. to form a mixture;
(3) optionally, filtering the mixture of the step-(2) to obtain a filtrate;
(4) cooling the mixture of the step-(2) or the filtrate of the step-(3) to a temperature of below about 30° C.; and
(5) isolating the crystalline relugolix from the mixture obtained in the step-(4), wherein the crystalline relugolix is characterized by XRPD spectrum having peak reflections as discussed supra.

In one embodiment, the present invention provides a process for the preparation of crystalline relugolix, the process comprising the steps of
(1) providing a solution of relugolix in dimethylacetamide at a temperature of about 40° C. to about 45° C.;
(2) combining the solution of the step-(1) with C₁-C₆ alkyl alcohol at a temperature of about 40° C. to about 45° C. to form a mixture;
(3) optionally, filtering the mixture of the step-(2) to obtain a filtrate;
(4) cooling the mixture of the step-(2) or the filtrate of the step-(3) to a temperature of below about 30° C.; and
(5) isolating the crystalline relugolix from the mixture obtained in the step-(4), wherein the crystalline relugolix is characterized by XRPD spectrum having peak reflections as discussed supra.

In one embodiment, the present invention provides a process wherein relugolix is obtained in a purity of ≥99.5%, and wherein the level of impurities designated herein as impurity E, impurity F, impurity G, or the compound XV is less than 0.15% w/w relative to the amount of relugolix, the compound I, as determined by High Performance Liquid Chromatography (HPLC),

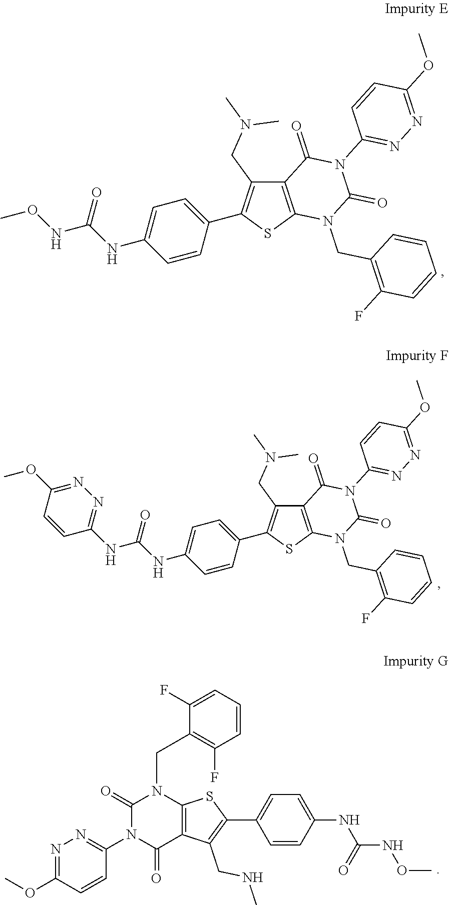

In one embodiment, relugolix, the compound I is obtained in a purity of ≥99.5%, and wherein the level of any of the impurity as described above is less than 0.15%

In one embodiment, relugolix, the compound I is obtained in a purity of ≥99.5% and wherein the level of any of the impurity as described above is less than 0.10%

In one embodiment, relugolix, the compound I is obtained in a purity of ≥99.5% and wherein the level of any of the impurity as described above is less than 0.05%

In one embodiment, relugolix, the compound I is obtained in a purity of ≥99.5% and wherein any of the impurity as described above is not detected.

In one embodiment, relugolix, the compound I, obtained by the process of the present invention is essentially free of nitrosamines impurities.

In one embodiment, the present invention provides relugolix wherein the level of nitrosamine impurity designated herein as impurity H is less than 0.15 ppm.

In one embodiment, relugolix obtained by the present invention is essentially free of nitrosamine impurity H.

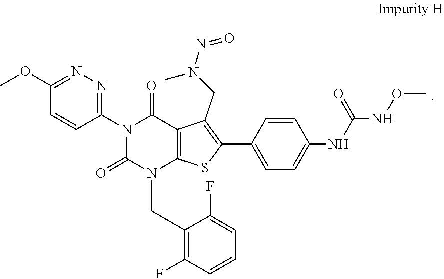

In one embodiment, the relugolix, the compound I, obtained by the processes herein described is in a crystalline form.

In one embodiment, the crystalline relugolix obtained by the process of the present invention as described herein above in one or more of the embodiments, is characterized by an X-ray powder diffraction (XRPD) spectrum having peak reflections at about 7.3, 8.9, 9.9, 12.0, 16.6, 17.3, 22.2, 22.7, and 27.4±0.2 degrees 2 theta.

In one embodiment, the present invention provides a pharmaceutically acceptable salt of relugolix, the compound I, with an organic acid or an inorganic acid.

In one embodiment, the organic acid is selected from the group consisting of oxalic acid, malic acid, maleic acid, malonic acid, tartaric acid, dibenzoyl tartaric acid, fumaric acid, citric acid, malic acid, succinic acid, mandelic acid, lactic acid, formic acid, acetic acid, salicylic acid, propionic acid, 2-chloromandelic acid, p-toluenesulfonic acid, ethane-1,2-disulfonic acid, camphor sulfonic acid, ethane sulfonic acid, methane sulfonic acid, naphthalene-2-sulfonic acid, benzene sulfonic acid, adipic acid, gluconic acid, glutaric acid, glutamic acid, palmitic acid, aspartic acid, and the like; and the inorganic acid is selected from the group consisting of hydrochloric acid, hydrobromic acid, sulfuric acid, nitric acid, phosphoric acid, and the like.

In one embodiment, the present invention provides a process for the preparation of a pharmaceutically acceptable salt of relugolix, the compound I, with an organic acid or an inorganic acid, the process comprising the steps of:
(a-ii) reacting relugolix, the compound I, with an organic acid or an inorganic acid to form a reaction mixture;
(b-ii) obtaining the pharmaceutically acceptable salt of relugolix with an organic acid or an inorganic acid from the reaction mixture of the step (a-ii); and
(c-ii) isolating the pharmaceutically acceptable salt of relugolix with an organic acid or an inorganic acid.

The organic acid and the inorganic acid used in the step (a-ii) in the preparation of the pharmaceutically acceptable salt of relugolix, the compound I, is as discussed supra.

In one embodiment, in the step (a-ii), the reaction of relugolix with an inorganic or organic acid may be carried out in presence of a solvent.

In one embodiment, the solvent includes but is not limited to esters such as methyl acetate, ethyl acetate, n-propyl acetate, isopropyl acetate, tert-butyl acetate and the like; hydrocarbons such as toluene, xylene, chlorobenzene, heptane, hexane and the like; ethers such as diethyl ether, diisopropyl ether, methyl tert-butyl ether, tetrahydrofuran, dioxane and the like; ketones such as acetone, ethyl methyl ketone, methyl isobutyl ketone and the like; alcohols such as methanol, ethanol, n-propyl alcohol, isopropyl alcohol, n-butyl alcohol, isobutyl alcohol, sec-butyl alcohol, tert-butyl alcohol, pentanol, octanol and the like; haloalkanes such as dichloromethane, chloroform, ethylene dichloride, and the like; dimethyl sulfoxide; dimethylacetamide; water; or mixtures thereof.

In one embodiment, in the step (a-ii), the reaction may be carried out at a temperature of about 0° C. to about 100° C.

In one embodiment, in the step (a-ii), the reaction may be stirred for a suitable time. The stirring time may range from about 30 minutes to about 10 hours, or longer.

In one embodiment, the step (b-ii) involving obtaining the pharmaceutically acceptable salt of relugolix from the reaction mixture of the step (a-ii) may be carried out by:
(1) optionally cooling, and stirring the mixture obtained in the step (a-ii); or
(2) removing the solvent from the mixture obtained in the step (a-ii); or
(3) treating the mixture of the step (a-ii) with an anti-solvent, optionally cooling and stirring the obtained mixture.

In one embodiment, the pharmaceutically acceptable salt of relugolix is obtained by optionally cooling and stirring the mixture of the step (a-ii). The stirring time may range from about 30 minutes to about 10 hours, or longer. The temperature may range from about 0° C. to about 30° C.

In one embodiment, the pharmaceutically acceptable salt of relugolix is obtained by removing the solvent from the mixture obtained in the step (a-ii). Removal of solvent may be accomplished by substantially complete evaporation of the solvent; or concentrating the solution, cooling the solution if required and filtering the obtained solid.

The solution may also be completely evaporated in, for example, a rotavapor, a vacuum paddle dryer or in a conventional reactor under vacuum above about 720 mm Hg.

In one embodiment, the pharmaceutically acceptable salt of relugolix is obtained by adding an anti-solvent to the mixture obtained in the step (a-ii) to form a mixture, and optionally cooling and stirring the obtained mixture. The stirring time may range from about 30 minutes to about 10 hours, or longer. The temperature may range from about 0° C. to about 30° C.

In one embodiment, the anti-solvent is selected such that the pharmaceutically acceptable salt of relugolix is precipitated out from the solution.

In one embodiment, the anti-solvent includes, but is not limited to esters such as methyl acetate, ethyl acetate, n-propyl acetate, isopropyl acetate, tert-butyl acetate and the like; hydrocarbons such as toluene, xylene, chlorobenzene, heptane, hexane and the like; ethers such as diethyl ether, diisopropyl ether, methyl tert-butyl ether, tetrahydrofuran, dioxane and the like; ketones such as acetone, ethyl methyl ketone, methyl isobutyl ketone and the like; alcohols such as methanol, ethanol, n-propyl alcohol, isopropyl alcohol, n-butyl alcohol, isobutyl alcohol, sec-butyl alcohol, tert-butyl alcohol, pentanol, octanol and the like; haloalkanes such as dichloromethane, chloroform, ethylene dichloride, and the like; dimethyl sulfoxide; dimethylacetamide; water; or mixtures thereof.

In one embodiments, in the step (c-ii) of the process, the pharmaceutically acceptable salt of relugolix is isolated by any method known in the art. The method, may involve any of techniques, known in the art, including filtration by gravity or by suction, centrifugation, and the like.

In one embodiment, the isolated pharmaceutically acceptable salt of relugolix may be further dried. The drying may be carried out at temperature from about room temperature to about 100° C. with or without vacuum. The drying may be carried out for any desired time until the required product quality is achieved. The drying time may vary from about 1 hour to about 25 hours, or longer.

In one embodiment, the pharmaceutically acceptable salt of relugolix as described herein is converted to relugolix free base by treating the pharmaceutically acceptable salt of relugolix with a base.

In one embodiment, the base is selected from the group consisting of sodium hydroxide, potassium hydroxide, lithium hydroxide, ammonium hydroxide, calcium hydroxide, magnesium hydroxide, sodium carbonate, potassium carbonate, lithium carbonate, ammonium carbonate, ammonium bicarbonate, sodium bicarbonate, potassium bicarbonate and lithium bicarbonate.

In one embodiment, the present invention provides a process for the purification of relugolix, the process comprising the steps of.
(a-iii) reacting relugolix with an organic acid or an inorganic acid to obtain a pharmaceutically acceptable salt of relugolix; and
(b-iii) reacting the pharmaceutically acceptable salt of relugolix as obtained in the step (a-iii) with a base to give relugolix.

The organic acid and the inorganic acid used in the step (a-iii) and the base used in the step (b-iii) are as discussed supra.

In one embodiment, the present invention provides pharmaceutical compositions comprising relugolix obtained by the processes described herein, having a D₉₀ particle size of less than about 250 microns, preferably less than about 150 microns, more preferably less than about 50 microns, still more preferably less than about 20 microns, still more preferably less than about 15 microns, and most preferably less than about 10 microns.

In one embodiment, the present invention provides pharmaceutical compositions comprising relugolix obtained by the processes described herein, having a D50 particle size of less than about 250 microns, preferably less than about 150 microns, more preferably less than about 50 microns, still more preferably less than about 20 microns, still more preferably less than about 15 microns, and most preferably less than about 10 microns.

The particle size described herein can be obtained by, for example, any milling, grinding, micronizing or other particle size reduction method known in the art to bring the solid state relugolix into any of the foregoing desired particle size range.

The examples that follow are provided to enable one skilled in the art to practice the invention and are merely illustrative of the invention. The examples should not be read as limiting the scope of the invention.

### EXAMPLES

### Example 1: Preparation of Ethyl 2-[(ethoxycarbonyl) amino]-4-methyl-5-(4-nitrophenyl) thiophene-3-carboxylate (Compound XIII)

To a suspension of ethyl 2-amino-4-methyl-5-(4-nitrophenyl)thiophene-3-carboxylate (5 g) in toluene (25 mL) was added ethyl chloroformate (5.3 g) and the mixture was refluxed for about 5 hrs. The reaction mixture was concentrated in vacuo, co-distilled with methyl tert-butyl ether. Methyl tert-butyl ether was added to the residue and the resulting precipitate was filtered, washed with methyl tert-butyl ether and dried to obtain compound XIII as yellow powder (5.7 g).

Purity: 99.84% by HPLC

¹H NMR (CDCl₃): δ 1.35 (3H, t, J=7.1 Hz), 1.42 (3H, t, J=7.2 Hz), 2.42 (3H, s), 4.31 (2H, q, J=7.1 Hz), 4.39 (2H, q, J=7.2 Hz), 7.59 (2H, d, J=9.0 Hz), 8.27 (2H, d, J=9.0 Hz), 10.66 (1H, s).

IR (KBr): 1740, 1665, 1597, 1557, 1533, 1516, 1352, 1257 cm⁻¹

### Example 2: Preparation of Ethyl 2-[(ethoxycarbonyl) amino]-4-methyl-5-(4-nitrophenyl) thiophene-3-carboxylate (Compound XIII)

To ethyl 2-amino-4-methyl-5-(4-nitrophenyl) thiophene-3-carboxylate (5 g) was added ethyl chloroformate (5.3 g) and the mixture was maintained at about 95-105° C. for about 3 hr. The reaction mixture was concentrated in vacuo. Methyl tert-butyl ether was added to the residue and the resulting precipitate was filtered, washed with methyl tert-butyl ether and dried to obtain compound XIII as yellow powder (5.8 g, 94%). Purity: 99.84% by HPLC.

### Example 3: Preparation of Ethyl 2-{[(2,6-Difluorophenyl) methyl](ethoxycarbonyl)amino}-4-methyl-5-(4-nitrophenyl) thiophene-3-carboxylate (Compound XII)

To a solution of ethyl 2-[(ethoxycarbonyl) amino]-4-methyl-5-(4-nitrophenyl) thiophene-3-carboxylate (5.5 g) in dimethylformamide (27 mL) was added potassium iodide (2.6 g) and potassium carbonate (2.2 g). A solution of 2,6-difluorobenzyl chloride (2.6 g) in dimethylformamide was then added to the reaction mixture. The reaction mixture was stirred at room temperature for about 48 hrs, the resulting precipitate was filtered off and the filtrate was concentrated in vacuo. The residue was diluted with ethyl acetate and water. The resulting extract was washed with water, concentrated in vacuo, co-distilled the residue with methyl tert-butyl ether. Methyl tert-butyl ether was added to the residue and the resulting precipitate was filtered, washed with methyl tert-butyl ether and dried to obtain compound XII as a yellow powder (6.6 g). Purity: 99.63% by HPLC.

¹HNMR (CDCl₃): δ 1.15-1.35 (6H, m), 2.40 (3H, s), 4.15-4.29 (4H, m), 4.97 (2H, s), 6.86 (2H, t, J=7.8 Hz), 7.25-7.32 (1H, m), 7.51 (2H, d, J=8.8 Hz), 8.25 (2H, d, J=8.8 Hz).

IR (KBr): 1717, 1597, 1524, 1475, 1392, 1348 cm⁻¹.

### Example 4: Preparation of ethyl 4-(bromomethyl)-2-{[(2,6-difluorophenyl) methyl](ethoxycarbonyl) amino}-5-(4-nitrophenyl) thiophene-3-carboxylate (Compound XI)

A mixture of Ethyl 2-{[(2,6-difluorophenyl) methyl](ethoxycarbonyl)amino}-4-methyl-5-(4-nitrophenyl) thiophene-3-carboxylate (5 g), N-bromosuccinimide (2.5 g) and azobisisobutyronitrile (1.9 g) in ethyl acetate (30 mL) was refluxed for about 5 hrs. The reaction was cooled to about room temperature and water was added. Both layers were separated and aqueous layer was extracted with ethyl acetate. Organic layer was washed with water, dried and concentrated in vacuo to obtain residue. Heptane and toluene were added to the obtained residue, resulting precipitate was filtered, washed with heptane and dried to obtain compound XI as a yellow solid (5.2 g, 91%). Purity: 92.67% by HPLC.

¹H NMR (CDCl₃): δ 1.15-1.39 (6H, m), 4.09-4.39 (4H, m), 4.71 (2H, s), 4.99 (2H, s), 6.86 (2H, t, J=7.8 Hz), 7.22-7.32 (1H, m), 7.72 (2H, d, J=8.0 Hz), 8.32 (2H, d, J=8.0 Hz).

IR (KBr): 1725, 1628, 1522, 1475, 1379, 1348 cm⁻¹.

### Example 4: Preparation of ethyl 2-{[(2,6-Difluorophenyl) methyl](ethoxycarbonyl)amino}-4-{1[(2-methoxyethyl) (methyl)amino]methyl}-5-(4-nitrophenyl) thiophene-3-carboxylate (Compound X)

To a solution of ethyl 4-(bromomethyl)-2-{[(2,6-difluorophenyl) methyl](ethoxycarbonyl) amino}-5-(4-nitrophenyl) thiophene-3-carboxylate (4.9 g) in dimethyl formamide (9.8 mL) was added a mixture of N, N-diisopropylethylamine (1.3 g) and N-(2-methoxylethyl) methylamine (0.8 g) in a dropwise manner. The reaction mixture was stirred at about room temperature for about 4 hrs and then concentrated in vacuo. The residue was diluted with methyl tert-butyl ether and water, concentrated hydrochloric acid was added at about 10-15° C. The mixture was stirred, layers were separated and aqueous layer was extracted twice with ethyl acetate. The extract was washed with saturated sodium bicarbonate solution and brine, concentrated in vacuo and co-distilled with heptane. Heptane was added to the residue and resulting precipitate was filtered, washed with heptane and dried to obtain compound X as a yellow solid (3.5 g, 70%)).

Purity: 97.04% by HPLC.

¹H NMR (CDCl₃): δ 1.15-1.30 (3H, m), 1.31 (3H, t, J=7.4 Hz), 2.11 (3H, s), 2.40-2.60 (2H, m), 3.28 (2H, s), 3.30-3.45 (2H, m), 3.68 (2H, s), 4.10-4.30 (2H, m), 4.23 (2H, q, J=7.4 Hz), 5.01 (2H, s), 6.85 (2H, t, J=7.6 Hz), 7.20-7.30 (1H, m), 7.69 (2H, d, J=8.8 Hz), 8.24 (2H, d, J=8.8 Hz).

### Example 5: Preparation of 2-{[(2,6-Difluorophenyl) methyl](ethoxycarbonyl)amino}-5-{4-{1[(methoxycarbamoyl) amino]phenyl}-4-1[(2-methoxyethyl) (methyl)amino]methyl}thiophene-3-carboxylic Acid [Compound VII]

### Step 1: Preparation of ethyl-5-(4-aminophenyl)-2-{[(2,6-difluorophenyl) methyl](ethoxycarbonyl)amino}-4-{1[(2-methoxyethyl) (methyl)amino]methyl}thiophene-3-carboxylate (Compound IX)

To the stirred solution of ethyl 2-{[(2,6-difluorophenyl) methyl](ethoxycarbonyl)amino}-4-{[(2-methoxyethyl) (methyl) amino]methyl}-5-(4-nitrophenyl) thiophene-3-carboxylate (5 g) and ethanol (25 mL) was added acetic acid (1 mL) and hydrogenated over 10% palladium on carbon (50% wet, 0.35 g) under hydrogen pressure at about 50-60° C. for about 3-4 hrs. After completion of reaction, reaction mixture was filtered through celite and washed with ethanol. Filtrate was concentrated under reduced pressure, obtained residue was diluted with water and methyl tert-butyl ether, and stirred. Layers were separated, saturated sodium bicarbonate was added to the aqueous layer. Aqueous layer was extracted in ethyl acetate and concentrated under reduced pressure to yield compound IX as an oil. Purity: 97.32% by HPLC.

### Step 2: Preparation of ethyl 2-{[(2,6-difluorophenyl) methyl](ethoxycarbonyl) amino}-5-{4-[(methoxycarbamoyl)amino]phenyl}-4-{[(2-methoxyethyl) (methyl)amino]methyl}thiophene-3-carboxylate (Compound VIII)

To the stirred solution of 1, 1′-carbonyldiimidazole (2.7 g), acetonitrile (15 mL) and triethylamine (2.6 mL) was added methoxyamine hydrochloride (1.4 g) at about 5-15° C. The reaction mixture was warmed to internal temperature of about 20-30° C. and stirred for about 2-3 hr. The solution of compound IX in acetonitrile (10 mL) was added thereto under stirring. The reaction mixture was warmed to about 50-60° C. and stirred for about 2-3 hr. After completion of reaction, distill out solvent from reaction mixture under reduced pressure and reaction mixture was diluted with water and ethyl acetate, and stirred. Both layers were separated and ethyl acetate layer was washed with 10% sodium chloride solution and concentrated under reduced pressure to obtain compound VIII as an oil. Purity: 96.53% by HPLC.

### Step 3: Preparation of 2-{[(2,6-difluorophenyl) methyl](ethoxycarbonyl) amino}-5-{4-[(methoxycarbamoyl)amino]phenyl}-4-{[(2-ethoxyethyl) (methyl) amino]methyl}thiophene-3-carboxylic acid (Compound VII)

To the stirred solution of compound VIII in ethanol (10 mL) was added aqueous sodium hydroxide solution (0.9 g sodium hydroxide in 10 mL of water). The reaction mixture was warmed to internal temperature of about 50-60° C. and stirred for about 3-4 hr. After completion of reaction, reaction mixture was diluted with water and methyl iso-butyl ketone, and stirred. Both layers were separated and concentrated hydrochloric acid was added to the aqueous layer (pH 4-6) and product was extracted in methylene dichloride (25 mL) and concentrated under reduced pressure. Heptane was added and again concentrated under reduced pressure. Further heptane was added, stirred for about 4-5 hr, filtered, washed with heptane and dried under reduced pressure for about 4-5 hr to yield compound VII as yellow solid (4.4 g, 85%).

Purity: 95.35% by HPLC).

¹H NMR (CDCl₃): δ 1.17 (3H, br s), 2.45 (3H, s), 2.81 (2H, br s), 3.28 (3H, s), 3.55 (2H, t, J=4.8 Hz), 3.82 (3H, s), 3.92 (2H, s), 4.10-4.35 (2H, m), 5.06 (2H, s), 6.82 (2H, t, J=7.8 Hz), 7.16 (2H, d, J=8.4 Hz), 7.22-7.35 (1H, m), 7.60 (2H, d, J=8.4 Hz), 8.00-8.50 (2H, br). IR (KBr): 1713, 1605, 1528, 1472, 1408 cm⁻¹. LC-MS m/z: 607.35 [M+H⁺].

### Example 6: Preparation of N-(4-{1-[(2,6-difluorophenyl) methyl]-5-{[(2-methoxyethyl) (methyl)amino]methyl}-3-(6-methoxypyridazin-3-yl)-2,4-dioxo-1,2,3,4-tetrahydrothieno[2,3-d]pyrimidin-6-yl}phenyl)-N-methoxyurea (Compound III)

### Step 1: Preparation of Carbamic acid, [(2,6-difluorophenyl) methyl][5-[4-[[(methoxyamino)carbonyl]amino]phenyl]-4-[[(2-methoxyethyl)methylamino]methyl]-3-[[(6-methoxy-3-pyridazinyl)amino]carbonyl]-2-thienyl]-, ethyl ester (Compound V)

Under a nitrogen atmosphere, to the stirred solution of compound VII (5 g) and 3-amino-6-methoxypyridazine (Compound VI, 3 g) in methylene dichloride (15 mL) and dimethylformamide (10 mL) was slowly added 50% propylphosphonic anhydride (T₃P) ethyl acetate solution (10 g) at internal temperature of about 25-35° C. The reaction mixture was stirred at the same temperature for about 1-2 hr. After completion of reaction, methylene dichloride and water was added to the reaction mixture. The organic layer was separated, and the aqueous layer was extracted with methylene dichloride. The combined extracts of methylene dichloride layers were washed with sodium bicarbonate solution (2 g sodium bicarbonate in 25 mL water) and sodium chloride solution (1.1 g in 25 mL water) twice. Organic layer was dried over sodium sulphate and concentrated under reduced pressure, added methanol and concentrated under reduced pressure to yield Compound V.

### Step 2: Preparation of N-(4-{1-[(2,6-difluorophenyl) methyl]-5-{[(2-methoxyethyl) (methyl)amino]methyl}-3-(6-methoxypyridazin-3-yl)-2,4-dioxo-1,2,3,4-tetrahydrothieno[2,3-d]pyrimidin-6-yl}phenyl)-N-methoxyurea (Compound III)

Compound V was dissolved in methanol (10 mL) and sodium methoxide (0.14 g) was added. The mixture was stirred at internal temperature of about 25-35° C. for about 2 hr, added ethanol (15 mL) and again stirred at about the same temperature for about 2 hr. After completion of reaction, the reaction mixture was cooled and stirred at internal temperature of about 5-15° C. for about 1 hr. The solid was filtered, washed with ethanol and dried under reduced pressure for about 4-5 hr to yield compound III as a solid (3.5 g, 63%).

Purity: 95.45% by HPLC.

¹HNMR (CDCl₃): δ 2.13 (3H, s), 2.62 (2H, t, J=5.7 Hz), 3.26 (3H, s), 3.41 (2H, t, J=5.7 Hz), 3.74 (2H, br s), 3.82 (3H, s), 4.18 (3H, s), 5.32 (2H, br s), 6.92 (2H, t, J=8.3 Hz), 7.12 (1H, d, J=9.3 Hz), 7.29-7.35 (1H, m), 7.41 (2H, d, J=9.3 Hz), 7.54 (2H, d, J=9.0 Hz), 7.59 (2H, d, J=8.7 Hz), 7.66 (1H, s).

IR (KBr): 2936, 1717, 1674, 1591, 1530, 1460 cm⁻¹. LC-MS m/z: 668.1 [M+H⁺].

### Example 7: Preparation of Urea, N-[4-[5-(chloromethyl)-1-[(2,6-difluorophenyl)methyl]-1,2,3,4-tetrahydro-3-(6-methoxy-3-pyridazinyl)-2,4-dioxothieno[2,3-d]pyrimidin-6-yl]phenyl]-N′-methoxy-(Compound II)

To a solution of compound III (5 g) in tetrahydrofuran (50 mL) was added 1-chloroethyl chlorocarbonate (1 mL) at about −78° C. in a dropwise manner and reaction mixture was stirred at about room temperature for about hr. Reaction was monitored by TLC. Heptane was added to the reaction mass and the resulting precipitate was collected by filtration, washed with heptane, and dried to give Compound III as pale yellow to off white solid (4.1 g, 89%).

Purity: 90% by HPLC.

¹H NMR (CDCl₃): δ 3.83 (3H, s), 4.20 (3H, s), 4.81 (2H, brs), 5.32 (2H, brs), 6.98-6.93 (2H, t) 7.17-7.15 (1H, d), 7.38-7.33 (1H, m), 7.47-7.44 (1H, d), 7.55-7.53 (1H, d), 7.64-7.62 (1H, d), 7.72 (1H, s); LC-MS m/z: 615.32 [M+].

### Example 8: Preparation of Urea, N-[4-[5-(chloromethyl)-1-[(2,6-difluorophenyl)methyl]-1,2,3,4-tetrahydro-3-(6-methoxy-3-pyridazinyl)-2,4-dioxothieno[2,3-d]pyrimidin-6-yl]phenyl]-N′-methoxy-(Compound II)

To a solution of compound III (5 g), sodium bicarbonate (0.62 g) in tetrahydrofuran (60 mL) was added 1-chloroethyl chlorocarbonate (1.2 mL) at about −78° C. in a dropwise manner and reaction mixture was stirred at about room temperature for about 2 hr. Reaction was monitored by TLC. Water was added to the reaction mass and the resulting precipitate was collected by filtration, washed with water, and dried to give Compound II as pale yellow to off white solid (4.1 g, 89%).

Purity: 90% by HPLC.

### Purification of Compound II

Method 1: To a solution of above crude Compound II (1 g) in dimethylacetamide (7 mL) was added dropwise acetonitrile (21 mL) and stirred at about room temperature for about 24 hr. The resulting precipitate was collected by filtration, washed with acetonitrile, and dried to give Compound II as pale yellow to off-white solid (0.6 g, 60%). Purity: 97% by HPLC.

Method 2: To a solution of above crude Compound II (1 g) in dimethylacetamide (7 mL) was added dropwise methyl isobutyl ketone (21 mL) and stirred at about room temperature for about 24 hr. The resulting precipitate was collected by filtration, washed with acetonitrile, and dried to give Compound II as pale yellow to off-white solid (0.62 g, 62%).

Purity: 97% by HPLC.

Method 3: To a solution of above crude Compound II (1 g) in dimethylacetamide (7 mL) was added dropwise acetone (21 mL) and stirred at about room temperature for about 24 hr. The resulting precipitate was collected by filtration, washed with acetonitrile, and dried to give Compound II as pale yellow to off-white solid (0.45 g, 45%). Purity: 97% by HPLC.

### Example 9: Preparation of Relugolix

Compound II (7 g) was dissolved in dimethyl sulfoxide (21 mL). Then N,N-diisopropylethylamine (2.3 mL) followed by dimethylamine (2M in THF, 32.4 mL) were added dropwise at about 10-15° C. successively. After the mixture was stirred at about room temperature for about 30 min. Water was added to the reaction mixture and the whole mixture was extracted with ethyl acetate twice. The extract was washed with brine and concentrated in vacuo. Then methanol was added to the residue and the resulting precipitate was collected by filtration, washed with methanol, and dried to give relugolix (Compound I) as off white solid (4.5 g, 63%). Purity: 93.16% by HPLC. The solid was recrystallized from ethyl acetate-tetrahydrofuran to afford relugolix as colorless crystals.

¹H NMR (CDCl₃): δ 2.13 (6H, s), 3.68 (2H, s), 3.83 (3H, s), 3.96 (3H, s), 5.36 (2H, s), 6.8-7.0 (3H, m), 7.13 (1H, s), 7.2-7.4 (1H, m), 7.45-7.65 (6H, m), 8.10 (1H, d, J=2.6 Hz) LC-MS m/z: 624.0 [M+H⁺].

| | d- | Rel. |
|---|---|---|
| Pos. | spacing | Int. |
| [°2Th.] | [Å] | [%] |
| 7.41 | 11.92 | 23.84 |
| 8.96 | 9.86 | 63.73 |
| 9.95 | 8.88 | 52.30 |
| 10.25 | 8.62 | 11.09 |
| 11.00 | 8.03 | 2.06 |
| 11.38 | 7.77 | 32.76 |
| 12.10 | 7.31 | 43.46 |
| 12.41 | 7.12 | 12.59 |
| 13.15 | 6.73 | 12.01 |
| 13.41 | 6.59 | 8.57 |
| 14.88 | 5.95 | 24.45 |
| 15.73 | 5.63 | 5.12 |
| 15.95 | 5.55 | 6.28 |
| 16.45 | 5.38 | 82.92 |
| 16.63 | 5.32 | 100.00 |
| 17.32 | 5.11 | 75.98 |
| 17.48 | 5.07 | 22.16 |
| 18.02 | 4.92 | 15.76 |
| 18.69 | 4.74 | 27.17 |
| 18.85 | 4.70 | 17.25 |
| 19.38 | 4.57 | 31.23 |
| 20.00 | 4.43 | 22.74 |
| 20.15 | 4.40 | 15.63 |
| 20.64 | 4.30 | 4.45 |
| 21.22 | 4.18 | 11.75 |
| 21.33 | 4.17 | 12.19 |
| 21.81 | 4.07 | 41.39 |
| 21.97 | 4.04 | 44.24 |
| 22.22 | 3.99 | 49.25 |
| 22.56 | 3.93 | 27.13 |
| 22.76 | 3.90 | 51.73 |
| 22.96 | 3.87 | 29.00 |
| 23.51 | 3.77 | 19.21 |
| 24.38 | 3.64 | 11.45 |
| 25.01 | 3.55 | 7.75 |
| 25.24 | 3.52 | 5.6 |
| 25.51 | 3.48 | 4.69 |
| 26.12 | 3.40 | 11.25 |
| 26.57 | 3.35 | 28.04 |
| 27.40 | 3.25 | 23.22 |
| 27.77 | 3.20 | 8.07 |
| 28.07 | 3.17 | 6.87 |
| 28.31 | 3.14 | 5.96 |
| 28.98 | 3.07 | 22.22 |
| 29.67 | 3.00 | 13.99 |
| 30.24 | 2.95 | 7.11 |
| 31.06 | 2.87 | 7.37 |
| 31.33 | 2.85 | 5.07 |
| 32.00 | 2.79 | 3.78 |
| 32.68 | 2.73 | 13.77 |
| 34.08 | 2.62 | 5.91 |
| 34.74 | 2.57 | 8.00 |
| 35.31 | 2.53 | 3.55 |
| 36.52 | 2.45 | 4.49 |
| 37.29 | 2.40 | 3.86 |
| 37.95 | 2.36 | 2.39 |
| 38.30 | 2.34 | 1.90 |
| 39.79 | 2.26 | 3.42 |
| 40.06 | 2.24 | 2.42 |
| 41.08 | 2.19 | 1.95 |

### Example 10: Preparation of 2-((2,6-difluorobenzyl) (ethoxycarbonyl)amino)-4-((dimethylamino)methyl)-5-(4-(3-methoxyureido) phenyl) thiophene-3-carboxylic acid (Compound XVI)

To the stirred solution of ethyl 2-((2,6-difluorobenzyl) (ethoxycarbonyl)amino)-4-((dimethylamino) methyl)-5-(4-nitrophenyl) thiophene-3-carboxylate (50 g) and water (150 mL) was added acetic acid (50 mL) and hydrogenated over 5% palladium on carbon (50% wet, 1.5 g) under 2-3 kg/cm2 hydrogen pressure at about 65-70° C. for about 5 hr. After completion of reaction, reaction mixture was filtered through celite and washed with water. To the clear filtrate were added ethyl acetate and sodium bicarbonate under stirring. The two layers were separated, and the organic layer containing compound XVIII was used in the next step.

To the stirred solution of 1,1′-carbonyldiimidazole (25.15 g), ethyl acetate (100 mL) and triethylamine (7.85 g) was added methoxyamine hydrochloride (11.4 g) at about 5° C. to about 15° C. The reaction mixture was warmed to about room temperature and stirred for about 1 hr. The organic layer containing compound XVIII was added thereto under stirring. The reaction mixture was warmed to about 55° C. to about 60° C. and stirred for about 2 hr. After completion of reaction, added triethylamine (11.05 g) at about same temperature under stirring. After cooling the reaction mixture, water was added under stirring. The two layers were separated and the organic layer was concentrated under reduced pressure. Methanol was added and the mixture was concentrated to give a residue. To the residue, was added methanol (200 mL) and aqueous sodium hydroxide solution (9.1 g sodium hydroxide in 75 mL of water). The reaction mixture was warmed to temperature of about 55° C. to about 60° C. and stirred for about 6 hr. After completion of reaction, the reaction mixture was cooled to about below 25° C. and the pH of the reaction mixture was adjusted to about 6 to about 6.5 by using dilute hydrochloric acid. The reaction mass was concentrated under reduced pressure to obtain a residue. To the residue, was added water and dichloromethane, and the mixture was stirred for about 4-5 hr at about 5° C. to about 10° C., filtered, washed with water followed by dichloromethane. To the stirred solution of water, were added sodium hydroxide (7.3 g) and dichloromethane (175 mL). The pH of the reaction mixture was adjusted to about 6 to about 6.5 by using dil hydrochloric acid at about below 25° C. The reaction mixture was stirred for about 4-5 hr at about 5° C. to about 10° C., filtered, washed with water followed by dichloromethane. The solid was dried under reduced pressure for about 4 hr at 65-70° C. to yield 2-((2,6-difluorobenzyl) (ethoxycarbonyl)amino)-4-((dimethylamino)methyl)-5-(4-(3-methoxyureido) phenyl) thiophene-3-carboxylic acid as off white solid (39 g, 76%).

Purity: 99% by HPLC.

### Example 11: Preparation of ethyl (2,6-difluorobenzyl) (4-((dimethylamino)methyl)-3-((6-methoxypyridazin-3-yl) carbamoyl)-5-(4-(3-methoxyureido) phenyl) thiophen-2-yl) carbamate (Compound XV)

Under a nitrogen atmosphere, to the stirred solution of 2-((2,6-difluorobenzyl) (ethoxycarbonyl)amino)-4-((dimethylamino)methyl)-5-(4-(3-methoxyureido) phenyl) thiophene-3-carboxylic acid (30 g) and 3-amino-6-methoxypyridazine (8 g) in ethyl acetate (90 mL) and diisopropylethylamine (28.24 g) was slowly added 50% propylphosphonic anhydride (T₃P) ethyl acetate solution (59.35 g) at about 25° C. to about 35° C. The reaction mixture was stirred at about same temperature for about 3-4 hr. After completion of the reaction, sodium bicarbonate solution (1.5 g sodium bicarbonate dissolved in 90 mL water) was added and stirred for about 5-6 hr. The reaction mass was filtered, washed with water followed by ethyl acetate. Methanol was added to the wet cake followed by sodium bicarbonate solution (1.5 g sodium bicarbonate dissolved in 75 mL water) and the reaction mixture was stirred for about 1-2 hr. The reaction mass was filtered and washed with water followed by methanol. The solid was dried under reduced pressure for about 3-4 hr at about 70-80° C. to yield ethyl (2,6-difluorobenzyl) (4-((dimethylamino)methyl)-3-((6-methoxypyridazin-3-yl) carbamoyl)-5-(4-(3-methoxyureido) phenyl) thiophen-2-yl) carbamate as off-white solid (29 g, 81%).

Purity: 98.86% by HPLC.

### Example 12: Preparation of Relugolix

To the stirred solution of ethyl (2,6-difluorobenzyl) (4-((dimethylamino)methyl)-3-((6-methoxypyridazin-3-yl)carbamoyl)-5-(4-(3-methoxyureido) phenyl) thiophen-2-yl) carbamate (Compound XV, 10 g) in methanol (40 mL) was added 25% sodium methoxide solution (1.5 g). The mixture was stirred at about 55° C. to about 60° C. for about 2-3 hr. After completion of reaction, the reaction mixture was cooled and stirred at temperature of about 25° C. to about 35° C. for about 3-4 hr. The solid was filtered, washed with methanol (5 mL). Water was added to the wet cake and the mixture was stirred for about 1-2 hr. The reaction mass was filtered, washed with water followed by methanol. The solid was dried under reduced pressure for about 3-4 hr at about 70-80° C. to yield (1-(4-(1-(2,6-difluorobenzyl)-5-((dimethylamino) methyl)-3-(6-methoxypyridazin-3-yl)-2,4-dioxo-1,2,3,4-tetrahydrothieno[2,3-d]pyrimidin-6-yl) phenyl)-3-methoxyurea as off white solid (8 g, 86%).

Purity: 99.37% by HPLC.

XRD: 7.3, 8.9, 9.9, 12.0, 16.6, 17.3, 22.2, 22.7, and 27.4±0.2 degrees 2 theta

### Example 13: Preparation of Relugolix

1-(4-(1-(2,6-difluorobenzyl)-5-((dimethylamino) methyl)-3-(6-methoxypyridazin-3-yl)-2,4-dioxo-1,2,3,4-tetrahydrothieno[2,3-d]pyrimidin-6-yl) phenyl)-3-methoxyurea (3.5 g) in dimethylsulfoxide (7 mL) was stirred at about 40° C. to about 45° C. Ethanol (56 mL) was added at about 40° C. to about 45° C. The reaction mixture was stirred at about room temperature for about 5-6 hr. The solid was filtered, washed with ethanol and dried under reduced pressure for about 8-9 hr to yield relugolix as off-white solid (3 g, 85%).

Purity: 99.66% by HPLC.

XRD: 7.3, 8.9, 9.9, 12.0, 16.6, 17.3, 22.2, 22.7, and 27.4±0.2 degrees 2 theta

D₁₀: 11 μm, D₅₀: 64 μm, D₉₀: 133 μm.

### Example 14: Preparation of Relugolix

1-(4-(1-(2,6-difluorobenzyl)-5-((dimethylamino) methyl)-3-(6-methoxypyridazin-3-yl)-2,4-dioxo-1,2,3,4-tetrahydrothieno[2,3-d]pyrimidin-6-yl) phenyl)-3-methoxyurea (5 g) in dimethylacetamide (12.5 mL) was stirred at about 40° C. to about 45° C. Ethanol (5 mL) was added to the mixture at about the same temperature. The reaction mass was filtered, washed with ethanol (5 mL). To the clear filtrate was added ethanol (70 mL) at about 40° C. to about 45° C. The reaction mixture was stirred at about room temperature for about 5-6 hr. The solid was filtered, washed with ethanol and dried under reduced pressure for about 8-9 hr to yield relugolix as off-white solid (4.3 g, 85%).

Purity: 99.78% by HPLC.

XRD: 7.3, 8.9, 9.9, 12.0, 16.6, 17.3, 22.2, 22.7, and 27.4±0.2 degrees 2 theta

### Example 15: Preparation of Relugolix

1-(4-(1-(2,6-difluorobenzyl)-5-((dimethylamino) methyl)-3-(6-methoxypyridazin-3-yl)-2,4-dioxo-1,2,3,4-tetrahydrothieno[2,3-d]pyrimidin-6-yl) phenyl)-3-methoxyurea (1 g) in dimethylacetamide (2.5 mL) was stirred at about 40° C. to about 45° C. 2-propanol (16 mL) was added to the mixture at about 40° C. to about 45° C. The reaction mixture was stirred at about room temperature for about 5-6 hr. The solid was filtered, washed with ethanol and dried under reduced pressure for about 8-9 hr to yield relugolix as off-white solid (0.9 g, 90%).

Purity: 99.71% by HPLC.

XRD: 7.3, 8.9, 9.9, 12.0, 16.6, 17.3, 22.2, 22.7, and 27.4±0.2 degrees 2 theta

### Example 16: Preparation of Relugolix

1-(4-(1-(2,6-difluorobenzyl)-5-((dimethylamino) methyl)-3-(6-methoxypyridazin-3-yl)-2,4-dioxo-1,2,3,4-tetrahydrothieno[2,3-d]pyrimidin-6-yl) phenyl)-3-methoxyurea (2 g) in dimethylacetamide (5 mL) was stirred at about 40° C. to about 45° C. Methanol (32 mL) was added to the mixture at about 40° C. to about 45° C. The reaction mixture was stirred at about room temperature for about 5-6 hr. The solid was filtered, washed with methanol and dried under reduced pressure for about 8-9 hr to yield relugolix as off-white solid (0.8 g, 40%).

Purity: 99.70% by HPLC.

XRD: 7.3, 8.9, 9.9, 12.0, 16.6, 17.3, 22.2, 22.7, and 27.4±0.2 degrees 2 theta

### Example 17: Preparation of Relugolix

1-(4-(1-(2,6-difluorobenzyl)-5-((dimethylamino) methyl)-3-(6-methoxypyridazin-3-yl)-2,4-dioxo-1,2,3,4-tetrahydrothieno[2,3-d]pyrimidin-6-yl) phenyl)-3-methoxyurea (3.5 g) was dissolved in dimethylacetamide (7.7 mL) at about 30° C. to about 40° C. Ethanol (7 mL) was added to the mixture at about the same temperature. The reaction mass was filtered, washed with mixture of dimethylacetamide (1.05 mL) and ethanol (3.5 mL) to obtain a filtrate. Ethanol (45.5 mL) was heated at about 40° C. to about 45° C. and added to the above filtrate at about 40° C. to about 45° C. The reaction mixture was stirred at about room temperature for about 5-6 hr. The solid was filtered, washed with ethanol and dried under reduced pressure for about 8-9 hr to yield relugolix as off-white solid (3 g, 85%). Purity: 99.70% by HPLC.

XRD: 7.3, 8.9, 9.9, 12.0, 16.6, 17.3, 22.2, 22.7, and 27.4±0.2 degrees 2 theta

1. A process for the preparation of relugolix, a compound of formula I (the “compound I”) or a pharmaceutically acceptable salt thereof, 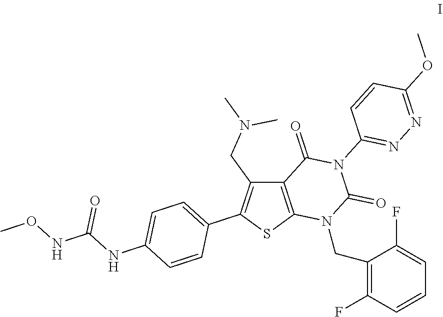 [SMILES: CONC(=O)Nc1ccc(-c2sc3c(c2CN(C)C)c(=O)n(-c2ccc(OC)nn2)c(=O)n3Cc2c(F)cccc2F)cc1]
the process comprising the steps of:
(a) reduction of a compound of formula XIX (the “compound XIX”) to obtain a compound of formula XVIII (the “compound XVIII”); 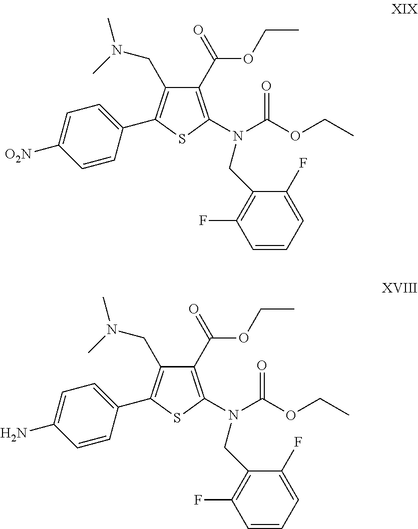 [SMILES: CCOC(=O)c1c(N(Cc2c(F)cccc2F)C(=O)OCC)sc(-c2ccc([N+](=O)[O-])cc2)c1CN(C)C ; CCOC(=O)c1c(N(Cc2c(F)cccc2F)C(=O)OCC)sc(-c2ccc(N)cc2)c1CN(C)C]
(b) reacting the compound XVIII with 1,1′-carbonyldiimidazole or a salt thereof, and methoxyamine or a salt thereof to obtain a compound of formula XVII (the “compound XVII”); 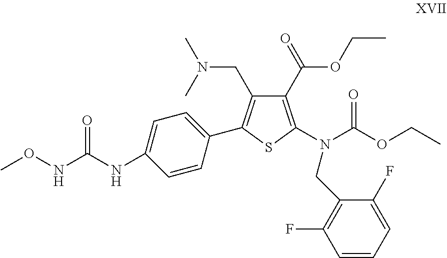 [SMILES: CCOC(=O)c1c(N(Cc2c(F)cccc2F)C(=O)OCC)sc(-c2ccc(NC(=O)NOC)cc2)c1CN(C)C]
(c) hydrolysing the compound XVII to obtain a compound of formula XVI (the “compound XVI”); 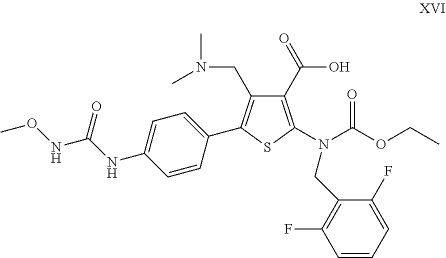 [SMILES: CCOC(=O)N(Cc1c(F)cccc1F)c1sc(-c2ccc(NC(=O)NOC)cc2)c(CN(C)C)c1C(=O)O]
(d) reacting the compound XVI with 3-amino-6-methoxypyridazine, a compound of formula VI (the “compound VI”) or a salt thereof, in the presence of a coupling agent to obtain a compound of formula XV (the “compound XV”); and 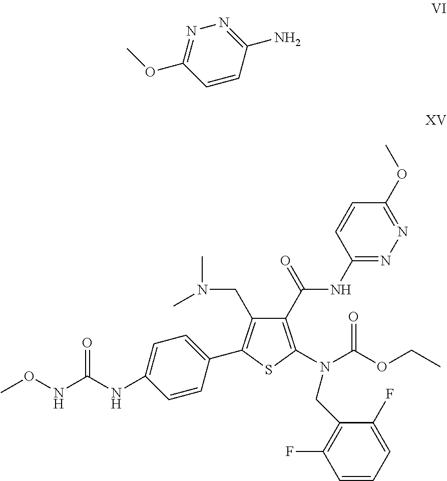
(e) cyclizing the compound XV in the presence of a base to obtain relugolix, the compound I, and optionally converting it to a pharmaceutically acceptable salt thereof;
wherein the compound XVIII obtained in step (a), without isolation, is carried forward for reaction in step (b).

2. The process of claim 1, wherein the reduction of compound (XIX) in the step (a) is carried out by hydrogenation of the compound XIX in the presence of a catalyst selected from the group consisting of palladium, platinum, Raney nickel, and a mixture thereof.

3. (canceled)

4. (canceled)

5. The process of claim 1, wherein the reduction of compound (XIX) in the step (a) is carried out in an aqueous medium.

6. The process of claim 1, wherein the reduction of compound (XIX) in the step (a) further comprises addition of an organic acid selected from the group consisting of formic acid, acetic acid, propionic acid, butanoic acid, trifluoroacetic acid, phthalic acid, fumaric acid, oxalic acid, tartaric acid, maleic acid, citric acid, succinic acid, malic acid, methanesulfonic acid, benzenesulfonic acid or p-toluenesulfonic acid and a mixture thereof.

7. (canceled)

8. The process of claim 1, wherein in the step (a), a biphasic reaction mixture is generated by adding a water immiscible organic solvent to the reaction mixture obtained after reduction, wherein the water immiscible organic solvent is selected from the group consisting of an ester solvent, a haloalkane solvent, a hydrocarbon and a mixture thereof.

9. (canceled)

10. The process of claim 8, wherein the ester solvent is selected from methyl acetate, ethyl acetate, n-propyl acetate, or tert-butyl acetate; and wherein the haloalkane solvent is selected from dichloromethane, chloroform, or dichloroethane.

11. (canceled)

12. The process of claim 8, wherein the biphasic reaction mixture is treated with a base, wherein the base is selected from the group consisting of lithium carbonate, sodium carbonate, potassium carbonate, sodium bicarbonate, potassium bicarbonate, sodium hydroxide, potassium hydroxide, and a mixture thereof.

13. (canceled)

14. The process of claim 12, wherein after treatment of the biphasic reaction mixture with the base, the biphasic reaction mixture is subjected to layer separation to obtain an organic layer and an aqueous layer, wherein the organic layer contains the compound XVIII.

15. The process of claim 14, wherein the organic layer containing the compound XVIII is carried forward as such for further reaction in the step (b).

16. The process of claim 1, wherein the hydrolysis reaction of the step (c) is carried out in the presence of a base to obtain the compound XVI, wherein the base is selected from the group consisting of sodium hydroxide, potassium hydroxide, lithium hydroxide, aqueous ammonia, and a mixture thereof.

17. (canceled)

18. The process of claim 1, wherein the compound XVI obtained in the step (c) is subjected to an base-acid treatment.

19. The process of claim 18, wherein the base is selected from the group consisting of sodium hydroxide, potassium hydroxide, lithium hydroxide, aqueous ammonia, and a mixture thereof; and wherein the acid is selected from the group consisting of hydrochloric acid, acetic acid and a mixture thereof.

20. (canceled)

21. The process of claim 1, wherein in the step (d), the coupling agent is selected from the group consisting of EDCI (N-(3-dimethylaminopropyl)-N′-ethylcarbodiimide hydrochloride), DCC (dicyclohexylcarbodiimide), DIC (diisopropylcarbodiimide), T₃P (propylphosphonic anhydride), HOBt (hydroxybenzotriazole hydrate), Oxyma (ethyl (hydroxyimino)cyanoacetate), and a mixture thereof.

22. The process of claim 1, wherein in the step (d), the reaction of the compound XVI with the compound VI is carried out in the presence of a base selected from the group consisting of diisopropylethylamine, triethylamine, pyridine, lutidine DMAP (4-(dimethylamino)pyridine), lithium carbonate, sodium carbonate, potassium carbonate, sodium bicarbonate, potassium bicarbonate, and a mixture thereof.

23. The process of claim 1, wherein in the step (e), the base is selected from the group consisting of sodium hydroxide, potassium hydroxide, lithium hydroxide, sodium methoxide, potassium methoxide, aqueous ammonia and a mixture thereof.

24. A process for the preparation of crystalline relugolix, the process comprising:
(1) providing a solution of relugolix in dimethylacetamide;
(2) combining the solution of the step (1) with an alcohol solvent to form a mixture;
(3) optionally, filtering the mixture of the step (2) to obtain a filtrate;
(4) obtaining crystalline relugolix from the mixture of the step (2) or the filtrate of the step (3); and
(5) isolating the crystalline relugolix, wherein the crystalline relugolix is characterized by an X-ray powder diffraction (XRPD) spectrum having peak reflections at about 7.3, 8.9, 9.9, 12.0, 16.6, 17.3, 22.2, 22.7, and 27.4±0.2 degrees 2 theta.

25. The process of claim 24, wherein the step (1), the step (2), and the step (3) are carried out at a temperature of about 35° C. to about 60° C.

26. The process of claim 24, wherein in the step (2), the alcohol solvent comprises a C₁-C₆ alkyl alcohol.

27. (canceled)

28. (canceled)

29. The process of claim 24, wherein in the step (4) of obtaining crystalline relugolix comprises cooling the mixture of the step (2) or the filtrate of the step (3), wherein the cooling is carried out to a temperature of below about 30° C.

30. (canceled)
